(19) United States
(12) Patent Application Publication
(10) Pub. No.: US 2003/0083241 A1
(43) Pub. Date: May 1, 2003

(54) Use of somatostatin receptor agonists in the treatment of human disorders of sleep hypoxia and oxygen deprivation

(76) Inventor: Charles W. Young, New York, NY (US)

Correspondence Address: FROMMER LAWRENCE & HAUG, 745 FIFTH AVENUE- 10TH FL., NEW YORK, NY, 10151, US

(21) Appl. No.: 10/280,517

(22) Filed: Oct. 25, 2002

Related U.S. Application Data
(60) Provisional application No. 60/336,512, filed on Nov. 1, 2001.
(60) Provisional application No. 60/331,866, filed on Nov. 20, 2001.

(51) Int. Cl. A61K 38/12

(52) U.S. Cl. 514/9

(57) ABSTRACT

The invention relates to a method of treating diverse human disorders that may arise, in part, out of sleep hypoxia and oxygen deprivation occurring in the context of sleep apnea/hypopnea disturbances. The disorders that may be treated by the invention comprise gastroesophageal reflux disease (GERD), asthma-associated gastroesophageal reflux (GER), GER-associated asthma, asthma, cardiomyopathy, cardioarrhythmia, congestive heart failure, sudden infant death syndrome, and diverse neurologic conditions. The mode of treatment uses somatostatin receptor ligands (SstRLs), particularly somatostatin-receptor agonists. The invention concerns the method of treatment utilizing, and compositions comprising SstRLs and somatostatin receptor agonists, including agonists of the somatostatin receptor types 2 and 5, particularly, the type 2A receptor (SsR-2A), including octreotide and lanreotide.

### FIELD OF INVENTION

The invention relates to a method of using somatostatin receptor agonists to treat diverse human disorders of sleep hypoxia and oxygen deprivation, including but not limited to: 1) gastroesophageal reflux disease (GERD), asthma-associated gastroesophageal reflux (GER), GER-associated asthma, and asthma; 2) obstructive sleep apnea (OSA), and OSA-associated conditions, including GER, asthma, cardiomyopathy, cardioarrhythmia, congestive heart failure, median nerve compression neuropathy (carpal tunnel syndrome) and cognitive impairment; as well as sleep apnea-associated sudden infant death syndrome (SIDS), 3) central sleep apnea (CSA), as well as CSA-associated conditions, including GER, cardiomyopathy, cardioarrhythmia, congestive heart failure, and cognitive impairment; 4) mixed pattern sleep apneas, including but not limited to post-vascular occlusion sleep apnea, dementia-associated sleep apnea, amyotrophic lateral sclerosis-associated sleep apnea, myasthenia gravis-associated sleep apnea, and alcoholism-related sleep apnea; 5) excess calpain-activation disorders in tissues where the injured cell population expresses somatostatin receptors; including, but not limited to the central nervous system, peripheral nerves, heart, liver, kidney, and gastrointestinal tract.

Various documents are cited in this text. Citations in the text can be by way of a citation to a document in the reference list, e.g., by way of an author(s) and document year, whereby full citation in the text is to a document that may or may not also be listed in the reference list.

There is no admission that any of the various documents cited in this text are prior art as to the present invention. Any document having as an author or inventor person or persons named as an inventor herein is a document that is not by another as to the inventor of entity herein. All documents cited in this text (“herein cited documents”) and all documents cited or referenced in herein cited documents are hereby incorporated herein by reference.

### BACKGROUND OF THE INVENTION

The invention relates, inter alia, to a novel method of using somatostatin receptor agonists, and other somatostatin-receptor ligands, particularly agonists of somatostatin receptor types 2A, for example, octreotide acetate and lanreotide to treat gastroesophageal reflux disease (GERD), and to treat various disorders of sleep hypoxia and oxygen deprivation. Such disorders may be clinically associated with gastroesophageal reflux (GER); including asthma, and respiratory disorders associated with sleep apnea, and, because of the association between chronic sleep apnea and its associated repeated occurrences of hypoxemia, cardiomyopathy and cardioarrhythmia, congestive heart failure, and peripheral and central nervous disorders, including cognitive impairment.

The invention encompasses novel compositions comprising somatostatin receptor agonists, and other somatostatin-receptor ligands, which compositions may be administered, for example, systemically, in appropriate dosages, by subcutaneous, intramuscular, intravenous, transdermal or transbuccal routes, orally, or by inhalation.

These disorders which may be treated by the novel method and compositions, include, but are not limited to, disorders affecting the gastrointestinal tract, for example, GERD and its complicating esophagitis, esophageal stricture, Barrett's esophagus, and adenocarcinoma of the esophagus and gastroesophageal junction, arising from, or occurring in the context of, Barrett's esophagus; disorders affecting both the gastrointestinal tract and the respiratory tract, for example, asthma-associated gastroesophageal reflux (GER) and GER-associated asthma and posterior laryngitis; and related disorders affecting the respiratory tract, for example, asthma; disorders affecting the central nervous system, the respiratory tract, and the gastrointestinal tract, for example, obstructive-, central-, or mixed-sleep apnea, with associated GER and/or asthma and cognitive impairment; and disorders affecting the central nervous system, respiratory system, and cardiovascular system, for example, obstructive-, central-, and mixed-sleep apnea with cardiomyopathy, and/or, cardioarrhythmia, and/or congestive heart failure.

In patients with GERD, the contents of the stomach repeatedly reflux into the esophagus, and to a lesser degree, the oropharynx and nasopharynx, with some entry into the respiratory tract. In addition to producing acute and chronic discomfort most-widely referred to as “heartburn”, these reflux episodes damage the esophagus to varying degrees, and to a lesser extent produce inflammation in the larynx and lungs. The esophageal injury presents as an inflammation, i.e., esophagitis, which may result in scarring with a strictural narrowing of the esophagus. The esophageal injury also presents in a more complex fashion, called “Barrett's esophagus”, wherein patches of columnar intestinal type epithelium appear, displacing the normally squamous epithelium of the esophagus. In a proportion of these patients “atypical” cells appear within the Barrett's epithelial areas; these atypical areas can undergo progressive change in appearance through “dysplasia” and then to cancer. Barrett's esophagus is considered to be a pre-cancerous condition for adenocarcinoma of the esophagus. The incidence of both Barrett's esophagus and adenocarcinoma of the esophagus has risen strikingly in the past 30 years, predominantly among men in Western societies.

In some individuals gastroesophageal reflux (GER) and asthma coexist; it has been estimated that from 40% to 80% of serious asthmatics also experience GER (asthma-associated GER). It is also known that episodes of gastroesophageal reflux can trigger acute asthmatic attacks (GER-associated asthma), and that, within a given patient, effective treatment of GER can have an ameliorative effect on asthmatic symptoms.

The tracheobronchial airways in asthmatic patients are characterized by hyperresponsiveness to multiple stimuli. Following exposure to relevant triggering stimuli, this hyperresponsiveness produces episodic narrowing of the air passages leading to acute respiratory difficulty. The “asthmatic” episodes are interspersed with prolonged periods wherein the patient is apparently free of symptoms; however, in many patients the general condition may be present for years and be associated with structural change within the airways that lead to permanent breathing limitation.

Although a scientific consensus has not been reached on the precise pathophysiologic mechanisms responsible for GERD and for the continuing increase in its frequency, in its most advanced manifestations three characteristics are evident: 1) an unduly relaxed lower esophageal sphincter (LES) at the basal state, 2) an increase in frequency of post-prandial (post-food intake) transient (complete) relaxation of the LES episodes (TLESRs), and 3) impaired esophageal motility, both with regard to intraluminal pressure and organized peristaltic waves. It appears likely that the earliest manifestations of GERD relate to an increase beyond the normal in TLESR events following food intake; this increase permits an increased level of exposure of the esophagus to gastric contents.

The subsequent further changes within the esophagus, and possibly the stomach and duodenum, are believed to be produced by the body's reaction to the injury, within the esophagus, caused by the repeated and increasing exposure to gastric contents, and in the more advanced cases to gastroduodenal contents.

The factors within the gastric fluid or duodenogastric fluid, that are collectively injurious to the esophagus, and to the respiratory tract in those patients in whom the reflux enters the pharynx, larynx and trachea, are: 1) gastric acid, 2) the pro-enzyme pepsinogen, that is converted and activated to proteolytic pepsin by gastric acid, and 3) some aspect of bile, most likely bile salts. The acute symptoms of chest pain and esophagitis correlate best with prolonged exposure to gastric acid at an acidic pH between 1.5 and 3.5. Traditional acid-inhibiting therapy, which decreases gastric acid secretion rates and raises the pH of gastric content to between 3.5 and 5.0, lessens the acute chest pain and decreases esophagitis in GERD patients, but does not correct the mucosal changes of Barrett's esophagus, nor produce improvement in the impaired esophageal motility or unduly relaxed LES.

The standard treatment of GERD varies with the intensity of symptoms experienced, in order of increasing aggressiveness, and it includes: 1) adjustments in eating habits, reducing the size and fat content of meals, 2) avoidance of the recumbent position for three hours following a meal, and particularly before going to bed for the night, 3) body weight reduction, 4) ingestion of antacids and antirefluxant mucosal protective agents, 5) reduction of the rate of gastric acid secretion by ingestion of histamine type-2 receptor antagonists, 6) more effective reduction of gastric acid secretion by ingestion of gastric acid proton pump inhibitors, 7) ingestion of prokinetic agents most commonly 5-HT-3 or 5-HT-4 agonists, which increase peristaltic activity within the esophagus and increase lower esophageal pressure, and 8) antireflux surgical therapy.

In patients in whom asthma and GER coexist, it has been suggested that the following factors in asthmatics may promote the occurrence of gastroesophageal reflux: 1) an autonomic dysregulation leading to heightened vagal responsiveness resulting in decreased basal LESP (“lower esophageal sphincter pressure”) and increased frequency of TLESRs, 2) increased intraabdominal pressure generated to overcome asthma-induced airflow obstruction, 3) airway hyperinflation flattening the crural diaphragm and making it a less effective contributor to LESP generation, 4) bronchodilator medications that produce broad relaxation of smooth muscle.

Similarly, it is proposed that GER episodes trigger acute asthmatic attacks by: 1) esophageal acid induced bronchoconstriction by way of a vagal-esophageal-bronchial reflex, 2) heightened bronchial reactivity, 3) microaspiration into the upper airway of refluxed esophageal contents.

Asthma is a condition wherein the airway is hyperreactive and reacts to certain inhaled stimuli with acute attacks characterized by increased secretion, swollen mucous membranes and intense bronchoconstriction of the airway smooth muscle. The disorder is also characterized by extensive infiltration with inflammatory cells, particularly by eosinophils. Asthma is characterized by an increase within the airway of a myriad of inflammatory mediators, among them the following are particularly relevant to the present invention: 1) Inducible nitric oxide synthase (NOS), 2) the cytokines tumor necrosis factor alpha (TNF-alpha), interleukin-1 beta, (IL-1 beta), and inferferon-gamma (INF-gamma), 3) the nuclear transcription factors NF-kappaB and AP-1, 4) endothelin-1(ET-1), and 5) the tachykinins substance P and Neurokinin A. All of these substances have physiologic functions in normal homeostasis; however, when produced in excess in patients with asthma, they contribute to the pathogenesis and progression of the illness.

The pathogenesis of asthma is multifactorial. In patients who develop the condition early in life the airway hyperreactivity appears to have an allergic or atopic basis, with the patient reacting to substances that do not produce a similar effect in the general population. In patients who have a late onset of asthma, not uncommonly in the context of an upper respiratory infection, the condition is considered “idiosyncratic” and precise allergens are difficult to identify. In both circumstances and in patients with a “mixed” pattern, the attacks can be triggered by a variety of stimuli, consistent with the general concept of airway hyperreactivity. Acute attacks may be triggered by known allergens, air pollution conditions, occupational factors, exposure to specific drugs, including aspirin and other non-selective non-steroidal anti-inflammatory drugs (NSAIDs), beta-adrenergic antagonists, physical exercise, and emotional stress. Hyperreactive airways are characterized by mucosa with increased numbers of inflammatory cells, particularly eosinophils, increased numbers of lymphocytes, and an increased capillary density. In acute attacks there is a reduction in the airway diameter due to the contraction of the bronchial smooth muscle, edema of the bronchial wall, and the presence of thick, tenacious secretions. The terminal airways are hyperinflated. In an asthma attack there is increased work in breathing due to the increased airway resistance. The decrease in expiratory volume and flow rates is associated with lower blood oxygen and elevated carbon dioxide levels.

Airway hyperreactivity is associated with and produced by the release of a myriad of inflammatory mediators as discussed above. Prominent among those are: histamine, serotonin, thromboxane A2, Leukotrienes B4, C4, D4, E4, bradykinin, substance P, endothelin-1 , reactive oxygen species, and nitric oxide. These mediators are synthesized and released from inflammatory cells, notably eosinophils, basophils, mast cells, neutrophils and macrophages. Their synthesis and release is triggered and regulated in part by a broad range of cytokines and lymphokines released from the inflammatory cells and from the infiltrating lymphocytes.

Traditional drug therapy of asthma has been based upon use of drugs that inhibit smooth muscle contraction and those which prevent or reverse inflammation, or stabilize mast cell membranes. More specific agents that inhibit the synthesis of specific inflammatory mediators, or block their peripheral action are under continuing development, and are progressively entering clinical use.

Among the smooth muscle relaxing agents, the adrenergic stimulants, including catecholamines, resorcinols, and saligenins produce smooth muscle relaxation by stimulating beta-adrenergic receptors with the activation of G proteins which act via cAMP and cAMP-activated protein kinases. The adrenergic stimulants are most broadly given by inhalation. Methylxanthines, i.e. theophylline and related compounds, given systemically, produce smooth muscle relaxation by an undefined mechanism. Anticholinergic agents, now usually non-absorbable and given by inhalation, block the bronchoconstrictive effects of vagally delivered acetylcholine. Glucocorticoids, given either systemically or by inhalation are the principal broad anti-inflammatory agents used in treating asthma. Cyclooxygenase non-selective non-steroidal anti-inflammatory agents are not used in treating asthma, because inhibition of cyclooxygenase-1 produces acute asthmatic attacks in some individuals, due to the joint withdrawal of prostaglandin E2, which has a bronchodilator effect, and an increase in the production of leukotrienes, which have strong bronchoconstrictor activity. Inhibitors of leukotriene synthesis, by blocking the enzyme 5-lipoxygenase, and antagonists of specific leukotriene receptors have recently entered broad clinical usage, blocking specified aspects of the inflammatory response. Cromolyn sodium and related salts are useful in treating asthma because they stabilize mast cell membranes against activation by a variety of stimuli. Activated mast cells release the bronchoconstrictive substances histamine and leukotrienes LTB4, LTC4 and LTD4.

It has been known that neuroendocrine cancers can release substances which produce bronchoconstriction, which can give the clinical appearance of asthma, and that somatostatin receptor agonists, e.g. octreotide, can relieve the bronchoconstrictive symptoms that are associated with carcinoids. However, these cases of “asthma” have been considered to be “atypical” asthma, and not true asthma. The collective understanding has been that octreotide only relieves cases of bronchoconstriction produced by the peripheral release of neuroendocrine substances, which travel to the lungs by the blood stream. Thus, it has been understood that octreotide therapy does not benefit patients with true asthma. Applicant, however, has by the instant invention, recognized the efficacy of compositions containing somatostatin receptor agonists, and related compounds, in the treatment of “true” asthma, and related conditions.

### SUMMARY AND OBJECTS OF THE INVENTION

1. Pathophysiologic Context of the Invention—GERD and Asthma

Applicant has recognized that the symptoms of both GERD and asthma occur in a diurnal context that is instructive with respect to the pathophysiology of the two disorders, and to multiple other disorders as well, and that an appropriate physiologic analysis of those diurnal relationships, expressed in the instant patent, provides guidance to a novel therapy for both GERD and asthma, and to multiple other disorders as well.

Sleep behavior influences the clinical pattern both in GERD and asthma patients; their symptoms commonly peak during sleep causing them to waken; in asthma patients with GER this applies to both the GER and the asthma symptoms (Fouad, et al., 1999; Peghini, et al., 1998; Farup, et al., 2001; Harding, 1999; Syabbalo, 1997). This symptom pattern is sufficiently common that the lifestyle adjustments and the specific treatments in both disorders are commonly adjusted to minimize nocturnal attacks to the degree possible.

Without wishing to necessarily be bound by any one particular theory, it is Applicant's belief that the primary underlying pathophysiologic abnormality in GERD, is excessive inhibitory neuronal signalling within the gastrointestinal tract, leading to esophageal dysmotility and a slack lower esophageal sphincter. Presently known treatments address this condition by the addition of pharmacologic stimulators of esophageal motility, rather than by removing the inhibitory molecules, or by blocking their biochemical effect.

Previously, somatostatin receptor agonists have been generally thought of as inhibitors of gut motility, because of their ability to slow diarrhea, and to calm upper abdominal symptoms in patients with intestinal obstruction. With that in mind, i.e., that somatostatin receptor agonists put the gut to sleep, those of skill in the art have been led away from the use of somatostatin receptor agonists for the treatment of GERD and related disorders. Applicant has, however, recognized that, by inhibiting the synthesis and release of, and the periperal actions of, the inhibitory neuronal signalling molecules within the esophagus, somatostatin receptor agonists actually stimulate esophageal motility and tighten the lower esophageal sphincter, and would therefor be particularly useful and suitable in the management of GERD and related disorders. This therapy is applicable even to patients with advanced GERD, in whom it has been commonly thought that the esophageal dysmotility and slack lower esophageal sphincter are the end stage of a chronic inflammatory process, induced by years of acid reflux wherein the relevant neural elements do not function because they have been destroyed by the illness itself. In this patient group further pharmacologic efforts have traditionally been abandoned in favor of protective surgery.

Applicant has further recognized that the nocturnal exacerbation of symptoms in these disorders can stem from the neuronal signaling pattern producing sleep in the given individual, and by a common characteristic of sleep, i.e., the reduction in blood oxygen levels as the “awake” respiratory pattern shifts to the respiratory pattern of sleep, which may be characterized by periods of apnea (cessation of respiration) of varying lengths. During apneic periods the level of oxygen in the airway, circulating blood and tissue progressively decreases and carbon dioxide levels rise. An association between GER episodes and periods of extended apnea has been recognized both in adults with obstructive sleep apnea, and in infants with a history of apparent life-threatening events (ALTE), referred to as a “near-miss” for sudden infant death syndrome (SIDS).(Ing, et al., 2000; See, 1989; Arad-Cohen, et al., 2000) Similarly, although not as well studied, sleep apnea-associated hypoxemia has been projected as a contributing factor in the timing of nocturnal asthmatic attacks.(Guilleminault, et al., 1988; Ballard, 1999) It is further of note that the obstructive sleep apnea syndrome, is commonly associated with drowsiness during the waking hours, leading to periods of sleep, and sleep apnea during the daytime.

Without wishing to be bound by any one particular theory, it is Applicant's belief that the primary underlying pathophysiologic gastrointestinal abnormality in patients with asthma-associated GER, is excessive inhibitory neuronal signalling within the esophageal musculature. Moreover, the Applicant has recognized that, by inhibiting the synthesis and release and peripheral actions of the inhibitory neuronal signalling molecules within the esophagus, treatment with somatostatin receptor agonists will stimulate esophageal motility and tighten the lower esophageal sphincter. This will ameliorate the symptoms of asthma-associated GER, and GER-associated asthma by reducing the frequency and severity of GER episodes in asthmatics, thereby reducing the frequency and severity of: 1) esophageal acid induced bronchoconstriction by way of a vagal-esophageal-bronchial reflex, 2) heightened bronchial reactivity, 3) microaspiration into the upper airway of refluxed esophageal contents. Moreover, by effects detailed in the following paragraphs, treatment with somatostatin receptor agonists, will have therapeutic effects upon asthmatic conditions, that are independent of these agents effects upon GER.

The instant invention comprehends, inter alia, that both the respective patterns of neuronal signaling producing sleep in affected individuals, and hypoxia occurring during sleep, associated with periods of apnea, obstructive, or of a central or mixed pattern, produce a biochemical change in the tissues of patients with GERD, and in patients with asthma, which further relaxes the smooth muscle of the esophagus and esophageal sphincter, without having a comparable effect on the bronchial smooth muscle.

The instant invention also provides that the pattern of nocturnal neuronal signaling in patients with active asthma contributes to the nocturnal increase in tissue inflammatory cells and to a nocturnal increase in the rate of production of reactive oxygen species (ROS) within the lungs, and that the hypoxia occurring during sleep in patients with symptomatic asthma, reduces the lung's capacity to produce protective substances against ROS, increasing further the extent of tissue damage. As one consequence of that damage, the inflammatory cells in the lung lose responsiveness to glucocorticoid therapy, resulting in an increase in asthmatic symptoms, which will be accompanied by an increased severity of hypoxemia. Accordingly, optimal therapy should attack not only the specific pathophysiologic abnormalities associated with the several disorders mentioned above, but also reduce the frequency and severity of chronic nocturnal hypoxic states and provide protection against the pathophysiologic consequences of hypoxia.

The instant invention recognizes, inter alia, that GERD is associated with a chronic increase, above the norm, in NANC inhibitory tone in the esophagus. At the level of the esophageal smooth muscle, that inhibitory tone is primarily produced by nitric oxide (NO.) generated from endothelial nitric oxide synthase (eNOS) within the gastrointestinal smooth muscles, triggered by vasoactive intestinal peptide (VIP) and pituitary adenylate cyclase activating peptide (PACAP) signaling through actions on the natriuretic peptide clearance receptor. VIP and PACAP also produce smooth muscle relaxation within the esophagus through adenylate cyclase activation via the VIP-1 and the PACAP-1 receptors.

Nitric oxide activates soluble guanylate cyclase (sGC) within the muscle cells, converting GTP to cGMP; the cGMP, in turn, activates guanylate kinase enzymes, which produce smooth muscle relaxation by selective phosphorylation of the muscle components. Adenylate cyclase converts ATP to cyclic-AMP (cAMP), which, in turn, activates adenylate kinase enzymes, which produce smooth muscle relaxation by selective phosphorylation of the muscle components.

In patients with GERD, this PACAP/VIP/nitric oxide synthase pathway appears to be upregulated chronically; it is further upregulated during sleep. It is the concept of the instant invention that in GERD patients, an exaggerated neuronal upregulation of PACAP and VIP expression and release, both within the brain and in the periphery drives the pathophysiologic process. This is consistent with published observations that circulating levels of VIP are increased both in patients with obstructive sleep apnea (Fischer and Jackowski, 1989), and in patients with Barrett's esophagus (Rossiter et al., 1991). It is also consistent with observations that PACAP and VIP actions promote REM sleep in rodents (Ahnaou, et al., 1999 and Jimenez-Anguiano, et al., 1996).

In addition, the sleep-associated decrease in breathing stimuli leads to a lowering of blood oxygen levels of varying degree. This graded hypoxemia is exemplified particularly in patients with obstructive sleep apnea (OSA), where GER is quite common (Ing et al., 2000). In OSA patients the apneic periods are associated with hypoxia and hypoxemia, which produce an upregulation of the enzyme heme oxygenase-1 (HO-1) within tissues (Chin, et al., 2001; Lee, et al., 1997). HO-1 carries out the reaction [iron protoporphyrin IX−→CO (carbon monoxide)+biliverdin+Fe++]. This enzymatic reaction helps the affected tissue survive under conditions of low oxygen because CO activates sGC in a manner analogous to that of NO., producing cGMP and relaxation of vascular smooth muscle. Moreover, the raised tissue levels of carbon monoxide make increased quantities of nitric oxide available for detoxification of reactive oxygen species, and for maintenance of smooth muscle relaxation (principally blood flow) by displacing the nitric oxide from heme tissue sites and slowing the degradation of nitric oxide, thus making it more available to the relevant enzyme target, soluble guanylate cyclase (Thorup, et al., 1999).

HO-1 and HO-2, a constitutive isoenzyme of HO-1, are present in the esophageal neuronal system, and the lower esophageal sphincter smooth muscle, where they contribute to the inhibitory neuronal signaling.(Ny, et al., 1995; Ny, et al., 1996; Xue, et al., 2000) The applicant has recognized that, by producing hypoxia within the esophageal smooth muscle and its associated neuronal sites, sleep apnea of sufficient duration increases level of inhibitory signals (increased availability of nitric oxide and carbon monoxide itself) within the esophagus and LES thereby producing symptomatic gastroesophageal reflux. Because, in contrast to nitric oxide, which is rapidly inactivated, carbon monoxide has a long duration within tissue, the increase in tissue inhibitory signaling persists after the hypoxic state has cleared with the resumption of breathing.

Accordingly, under hypoxic conditions, relaxation of gastrointestinal smooth muscle is further promoted by: high tissue levels of endogenous carbon monoxide, residual nitric oxide, and the PACAP and VIP engendered adenylate cyclase. These factors produce the nocturnal changes observed in GERD, i.e., a further decrease in lower esophageal sphincter pressure, esophageal dysmotility, and an increase in the frequency of transient lower esophageal sphincter relaxation events.

The other pathophysiologic events in GERD follow in sequence in the above-described context. A decreased esophagaeal motility and deceased basal lower esphophageal sphincter pressure (LESP) (especially at night) lead to gastroesophageal reflux of acid and peptic gastric secretions, thence to inflammation and the intestinalization of the epithelium, that is typical of Barrett's esophagus, and a further increase in dysmotility, and decrease in basal LESP. In time, bile reflux becomes a significant factor, possibly with the sphincter of Oddi, which permits entry of the contents of the common bile duct into the duodenum, becoming a participant in the sphincteric relaxation process.

Accordingly, in one embodiment of the instant invention, a therapy is provided which reduces the hypoxic episodes by changing the sleep patterns in asthmatic and GERD patients to reduce the frequency of sleep apneic episodes. In order to elucidate how the instant therapy produces the desired change in the abnormal sleep patterns that produce pathologic degrees of sleep apnea, the physiology and pathophysiology of sleep are discussed below in the section “Pathophysiologic Context of the Invention—Sleep Apneas”.

Patients with GER-associated asthma and asthma-associated GER probably constitute a mixed pathophysiologic group. Individual patients could range from those with GER with OSA in whom aspiration triggers acute respiratory attacks, but who do not have diffusely inflamed membranes and a hyperactive airway, to those with typical atopic asthma, with a hyperactive airway, who develop GER. The neurophysiologic basis for the GER events in the “true” asthmatic individuals may differ somewhat from that described in the paragraphs above. In contrast to the GERD patient where PACAP and VIP are considered plausible effectors for both sleep induction and gastroesophageal NANC neuronal signaling, TNF-alpha and IL-1 beta are both prominent in the asthma-associated lung inflammation; the two cytokines, which are produced in neurones in addition to inflammatory cells, are established somnogens. A coordinated CNS and peripheral nocturnal release of TNF-alpha and IL-1 beta, could provide the above-described sleep-associated increase in lung inflammation and an increase in NANC inhibitory tone within the gastrointestinal tract by a strong upregulation of iNOS.

Within the context of the above discussion, treatment of GER is achieved by use of a medication that decreases the frequency and severity of sleep apnea, and a treatment that decreases the excessive NANC inhibitory tone in the esophagus; both of these goals may be accomplished by reducing the excess neural secretion of VIP and PACAP in GERD patients, and by reducing the excess neural secretion of TNF-alpha and IL-1 beta in asthma patients, and by reducing, as well, their peripheral effects; this can be an object of the instant invention, which uses SstR-2A agonists to achieve that result. The instant invention considers also that the dose and timing of SstR-2A use may differ between the two treatment groups.

In non-inflamed lungs NANC inhibitory control of smooth muscle, both bronchial and vascular, is dependent upon both the adenylate cyclase and the guanylate cyclase pathways. VIP and PACAP, released from peptidergic nerves, produce bronchial relaxation by binding to their shared (adenylate cyclase-activating) receptors in bronchial muscle and blood vessels. Guanylate cyclase activation is dependent upon the diffusion of NO. from epithelial cell expression of inducible nitric oxide synthase, and endothelial cell nitric oxide synthase (eNOS) in the lung's blood vessels. In contrast to the status described previously with regard to the gastrointestinal smooth muscle, eNOS is not present in the bronchial smooth muscle (Feletou, et al., 2001). The natriuretic peptide clearance receptor interaction with eNOS, which greatly affects smooth muscle tone in the gastrointestinal tract, is not seen in lung tissue.

In non-inflamed lungs a reduction in neuronal release of VIP and PACAP does not disturb lung equilibrium, as indicated by the excellent general tolerance to SstR-2A agonists, including in patients with obstructive sleep apnea. However, lung airway nitric oxide production serves a dual function; although NO is an effective bronchodilator, particularly when combined with reduced glutathione as S-nitrosoglutathione, it may also serve an even more important role because it protects the lung by detoxifying reactive oxygen species (ROS) within the airway, notably including superoxide (Dweik et al., 2001).

Inflammation in asthmatic airways produces ROS at a high rate; although the elevated levels of nitric oxide produced in asthmatic airways convert highly toxic superoxide to the relatively non-toxic nitrate ion; levels of S-nitrosoglutathione are greatly decreased. Accordingly, the acutely inflamed asthmatic lung, where a major portion of the nitric oxide production is consumed by interaction with ROS, such as superoxide, and tissue levels of S-nitrosothiols are reduced, may be more dependent upon the adenylate cyclase-mediated bronchodilation mechanisms than is the case with non-inflammed lungs. In that circumstance an acute reduction in neuronal release of VIP and PACAP could have an acute deleterious effect. Because SstR-2A agonists inhibit the release of the cytokines TNF-alpha, IL-1 beta, and interferon gamma, and block their peripheral effects, they produce a progressive decrease in inflammation; accordingly, the instant invention envisions initiating their use in stable asthmatic patients and at low dosage, to avoid a major reduction in VIP and PACAP release until the drugs' anti-inflammatory effects have become evident.

2. Pathophysiologic Context of the Invention—Sleep Apneas

Sleep apnea syndromes fall into two basic classifications, 1) central sleep apnea (CSA) is relatively rare in the absence of congestive heart failure; however, a variant form of CSA, Cheyne-Stokes respiration (CSR-CSA), is occurs frequently in patients with congestive heart failure. 2) obstructive sleep apnea (OSA) is the far more common than CSA and has been more extensively studied. Some individuals may manifest a “mixed” sleep apnea syndrome with characteristics of both OSA and CSA. In the instant invention it is disclosed that therapy with somatostatin agonists will ameliorate symptoms of both CSA and OSA.

In individuals with CSA, and particularly the Cheyne-Stokes Respiration variant of CSA (CSR-CSA), the breathing pattern is characterized by alternating cycles of hyperventilation and apnea that occur most prominently during non-rapid eye movement sleep (NREM-sleep), rather than during rapid eye movement sleep (REM-sleep). The teaching of multiple research groups indicates that the cause of CSA, including CSR-CSA, is an increased chemosensitivity to the partial pressure of of arterial carbon dioxide, which, may produce during sleep a hyperventilatory response that lowers the arterial carbon dioxide below the apnea threshold, producing a central apnea in which there is no central signal to the respiratory muscles (Javaheri, 1999; Xie, et al., 2002; Leung and Bradley, 2001). The cause of the increased chemosensitivity to CO2 in individuals with idiopathic CSA is unknown; however, the teaching of Xie, et al., suggests that hypoxia itself, such as that occurring at in climbers at high altitude, or in patients with congestive heart failure, can narrow the difference between baseline arterial CO2 levels and the hypopnea/apnea threshold level for arterial CO2, thereby increasing the likelihood of ventilatory instability leading to episodes of CSA (Xie, et al., 2001). Although oxygen supplementation and/or increased CO2 in the inspired air may ameliorate symptoms of CSR-CSA, at present there is no standard treatment for the syndrome.

In contrast to the respiratory pattern of CSA described above, as taught by recent reviews on the topic, breathing patterns in OSA are, in some regards, an exaggeration of the normal pattern of respiration during sleep, wherein the respiratory volume/minute progressively decreases with a proportionate decrease in the arterial content of oxygen (paO2) and an increase in the arterial content of carbon dioxide (paCO2). The lowest respiratory volume/minute rates in normal individuals occur during rapid eye movement (REM-sleep), wherein there exists a paradox of high electrical activity and blood flow of the cortical areas of the brain (dreaming) and the greatest reduction of respiratory volume. Accordingly, in normal sleep paO2 levels are lower and paCO2 levels are higher during REM-sleep than they are during non-REM-sleep (NREM-sleep). Also in the course of normal REM-sleep, and to lesser degree in deep NREM-sleep, atonia of the postural muscles develops and extends to the muscles of the upper airway leading to a collapse of the soft tissues, resulting in upper airway obstruction of varying degree. The teaching of Werth et al., indicates that muscle atonia in non-REM sleep can be readily produced in normal individuals by selective REM sleep deprivation (Werth, et al., 2002).

In patients with obstructive sleep apnea/hypopnea syndromes the normal pattern of reduced ventilatory volumes becomes grossly exaggerated; respiratory minute volumes become progressively enfeebled, and, by reason of structural limitations on the airway configuration, on a genetic basis, or associated with obesity, or endocrine illnesses such as acromegaly, the airway collapse during muscle atonia becomes sufficient to block the upper airway. Normal levels of snoring become quite audible interrupted by pauses during which no airflow occurs; the affected individuals become variously hypoxic (Young, et al., 2002, Leung and Bradley, 2001; Krieger, et al., 1997). Although the central respiratory drive continues, it is for the most part too feeble to break the obstruction in the upper airway, that obstruction is broken when the individual partially wakens, the atonia is interrupted, the upper airway is momentarily cleared (Krieger, et al., 1997). After a varying period of time sleep resumes and the process is repeated. The repeated episodes of airway obstruction, hypoxia, and arousal, produce a sleep deprivation effect, leading to daytime sleepiness, and a continued disturbance of the sleep wake cycle (Leung and Bradley, 2001; Becker, et al., 1999; Krieger, et al., 1997).

Standard treatment for OSA is the use during sleep of a continuous positive airway pressure (CPAP) breathing apparatus, most commonly via a nasal mask. In concept, CPAP eliminates the negative pressure differential that produces collapse of the upper airway during muscular atonia; in individuals with OSA who are able to adapt to CPAP, this greatly reduces the hypoxia, permits more satisfactory sleep and breaks the recurrent apnea/hypopnea cycle (Flemons, 2002). Unfortunately, many individuals with OSA are unable to adapt to CPAP in its existing forms, leaving unfilled a significant medical need since, at present, there is no effective pharmacologic therapy for OSA (Hudgel and Thanakitcharu, 1998). It is the purpose of the instant invention to report that somatostatin agonists can ameliorate the symptoms of both CSA and OSA, respectively, through modifying the reactions of the peripheral chemoreceptors to hypoxia and carbon dioxide (CSA), and through altering the secretion, and actions of somnogenic molecules that set the body's circadian clock, and/or influence sleep through altering the sleep homeostat. Current teaching on the nature and control of the circadian clock and its interface with the sleep homeostat is presented in the following paragraphs.

As taught in recent reviews of the field, by Pando and Sassone-Corsi and by Dijk and Lockley, mammalian physiologic behavior, including the sleep-wake cycle, operates within the context of a central or “master” circadian clock located within the superchiasmatic nuclei (SCN) of the brain and a sleep homeostat, believed to be located outside of the SCN (Dijk and Lockley, 2002; Pando and Sassone-Corsi, 2001). Many peripheral tissues have circadian clocks also which receive input from the SCN clock. Adjustments of the central clock, referred to as “entrainment”, that accommodate to changing conditions of light and dark are made by light exposure of retinal ganglion cells which communicate with the SCN by the retinohypothalamic tract (RHT). In contrast, the teaching of Dijk and Lockley indicates that “the oscillation of the sleep homeostat is strongly, and maybe exclusively, determined by the sleep-wake cycle, wherein the awake state engenders the need to sleep, and sleep can be sustained for only a limited period before the awake state resumes (Dijk and Lockley, 2002). The above characteristic allows the use of light exposure to identify circadian neurotransmitters and sleep deprivation to identify endogenous substances which contribute to the control of the sleep homeostat.

The teaching of multiple laboratories, including that of Chen, et al., and Hannibal, et al., indicates that the primary neurotransmitter of the light signal is the aminoacid and neurotransmitter glutamate, but that PACAP, present along with glutamate in the retinal ganglion cells, modulates the effects of glutamate on a continuing basis (Chen, et al., 1999; Hannibal, et al., 1997). The subsequent teaching of Dziema and Obrietan, indicates that the PACAP modulation of glutamate effects occurs by PACAP-mediated effects on L-type calcium channel conductance in SCN neurons (Dziema and Obrietan, 2002). The combined glutamate PACAP signaling continues as long as light hits the retinal ganglion cells but the effects of light on the clock setting, i.e., a new entrainment differ depending upon their relationship to the light dark cycle currently embedded in the central clock.

In addition to the circadian elements discussed above, the sleep and wake cycle in mammals is known to be modulated by broad range of endogenous factors, prominent among which are melitonin, growth hormone releasing hormone (GHRH), and the cytokines interleukin-1 beta (IL-1beta), and TNF-alpha (Krueger, et al., 1999 and 2001; Takahashi, et al., 1999). The somnogenic actions of IL-1beta and TNF-alpha may be due, in part, to their activation within the brain of the heterodimeric transcription factor nuclear factor-kappaB (NF-kappaB), which itself upregulates mRNA transcription for IL-1beta and TNF-alpha. Chen et al., have demonstrated that NF-kappaB-like activity increases in murine cerebral cortex after sleep deprivation (Chen, Z., et al., 1999), and Kubota et al., have demonstrated that a peptide inhibitor of NF-kappaB inhibits spontaneous and IL-1beta-induced sleep in rats (Kubota et al., 2000). Other somnogens include PACAP (possibly independent of its circadian role described above), VIP, cortistatin and IL-6 (Ahnaou, et al., 1999 and 2000; Chen, et al., 1999; de Lecea, et al., 1996; Hannibal et al., 1997 and 2002; Jiminez-Anguiano, et al., 1996; Spier and de Lecea, 2000; Sutcliffe and de Lecea, 1999; Vgontzas, et al., 1997, 1999 and 2000).

The teaching of multiple laboratories indicates that TNF-alpha, IL-1beta, and NF-kappaB accumulate in brain of sleep deprived animals, and in the circulation of sleep-deprived humans (Chen, et al., 1999; Krueger, et al ., 2001; Takahashi, et al., 1999). These characteristics are consistent with those molecules being a contributing factor to “sleep debt” within the sleep homeostat. Although the narrowing of the upper airway produced by fat deposits, and the gross increase in weight of the chest wall evident in hyperobese individuals provides an intuitively clear explanation for the occurrence of OSA, the teaching of and others indicates that the fat deposits have metabolic effects as well, at least in part through their secretory products. The observation that hyperobese patients elevated blood levels of (fat cell-derived) TNF-alpha, and IL-6, provides a pathophysiologic context for these patients high rates of OSA; their secreted cytokines are adding to the sleep debt within the sleep homeostat.

In patients with OSA the development of muscle atonia is of particular relevance to the reduced respiratory muscle effort, and to the relaxation of the upper respiratory muscles which contributes to upper airway obstruction; the neuropharmacology of upper airway motor control has been extensively studied in animal model systems. The teaching of a number of groups in this regard has been reviewed recently by Kubin, et al., and by Homer (Kubin, et al., 1998; Horner, 2001). Current attention is focused upon the brain stem and the pontine regulation of rapid eye movement and muscle atonia. Models have been developed in rats and cats which permit the dissection of some of the neurotransmitter interactions. A standard model involves the injection of carbachol, a strong cholinergic agonist, into the pontine reticular formation (oral pontine reticular nucleus) producing thereby REM sleep and muscle atonia in the treated animal. The teaching of Ahnaou, et al., indicates that in rats positive interactions between muscarinic receptors for acetyl choline and receptors for PACAP enhanced REM sleep production; it was of particular interest that PACAP injections at this site could produce REM enhancement that lasted for several days (Ahnaou, et al., 2000). These observations indicate that PACAP can affect sleep patterns at two points, 1) modulating glutamate actions on entrainment of the central circadian clock, and 2) the pontine reticular formation where it interfaces with muscarinic signaling enhancing REM sleep and associated muscular atonia.

Without wishing necessarily to be bound by any one particular theory with respect to the ameliorative actions of somatostatin agonists upon sleep hypoxia, it is Applicants belief that the following published observations are germane to the ameliorative effect in individuals with OSA. Treatment with somatostatin agonists reduces secretion of neurotransmitters and cytokines which contribute to the accumulation of sleep debt in the sleep homeostat, eg. GHRH hormone secretion is inhibited by somatostatin and octreotide (Frieboes et al., 1997; Masuda et al., 1989; and Moller et al., 1989). Similarly, somatostatin and octreotide inhibit the secretion of TNF-alpha, Interleukin-1 beta and Interleukin-6, thereby reducing the activation of NF-kappaB within neuronal cells (Karalis, et al., 1994; Chao, et al., 1999; Lamrani, et al., 1999; Peluso, et al., 1996; Chowers, et al., 2000). Somatostatin agonists inhibit the secretion and the peripheral actions of PACAP by inhibitory effect on L-type calcium channels, and by blocking the G-protein mediated activation of adenylate cyclase (Zeng, et al., 1999; Athmann, et al., 2000).

Further, it is the Applicant's belief that the following published observations are germane to the amelioration of central sleep apnea (CSA) by somatostatin agonists. As discussed above, the signal pathophysiologic lesion in individuals with CSA is the development of a increased sensitivity within chemoreceptors to the arterial partial pressure of carbon dioxide, an event triggered at least in part by hypoxia. The teaching of Pederson et al., indicates that “the peripheral chemoreflex sensitivity to carbon dioxide is reduced by somatostatin, probably via the same mechanism as that by which somatostatin exerts its (inhibitory) effects on the ventilatory response to hypoxia” (Pedersen, et al., 1999).

It has been known for some years that obstructive sleep apnea occurring in patients with acromegaly, is ameliorated in many patients when octreotide is administered as therapy for the endocrine disorder (Barkan, 1997). Those skilled in the treatment of acromegaly, a disorder characterized by an increased expression and release of growth hormone and growth hormone releasing hormone in neoplastic cells, most commonly in adenomas of the pituitary gland, assumed that it was a specific effect limited to that disorder, and explained in part by the gross distortion of the tissues of the upper airway produced by the illness. It is the purpose of the Instant invention, to point out that the ameliorative effects of somatostatin agonists on sleep apnea syndromes extend beyond patients with acromegaly. 3. Pharmacologic Context of the Invention

The biology of somatostatin and somatostatin receptors, and the pharmacology and clinical use of somatostatin analogs, agonists and antagonists have been reviewed; among the most recent are those by Beglinger and Drew, 1999; Csaba and Dournaud, 2001; Patel, 1999; and Scarpignato and Pelosini, 1999 and 2001. In the peripheral nervous system, somatostatin (somatotropin release inhibiting factor=SRIF) is a modulator of endocrine and exocrine functions and regulates the differentiation and proliferation of normal and tumor cells. Within the nervous system somatostatin acts as a neuromodulator with physiological effets on neuroendocrine, motor and cognitive functions. Five somatostatin receptors have been cloned and termed sst1-sst5; sst2 mRNA gives rise to two protein isoforms, sst2A and sst2B.

Somatostatin receptors belong to the family of G protein-coupled receptors and bind the native peptides SRIF-14 and SRIF-28 with high affinity. Native SRIF receptors interact with different types of inhibitory G proteins. One of the most widely studied intracellular effectors is the adenylyl cyclase (AC)—cAMP—protein kinase A (PKA) pathway. In a broad variety of cell types, peripherally and in the central nervous system, SRIF inhibits basal and stimulated cAMP production.

Activation of somatostatin receptors also provides modulation of ion channels. Activation of potassium channels by somatostatin or somatostatin agonists results in cell hyperpolarization, which leads to a reduction of intracellular Ca⁺⁺ concentrations due to the inhibition of depolarization-induced calcium currents via volatage dependent calcium channels. Somatostatin also acts directly on voltage-gated Ca channels including those of the L- , N-, and P/Q-types. Stimulation of somatostatin receptors also activates protein phosphotyrosine phosphatases, an action that is believed to be responsible for inhibition of growth factor and cytokine signals by somatostatin receptor agonists.

Binding of agonists to somatostatin receptors leads to internalization of the receptors with progressive involvement with the cellular endosomal system and either recycling to the cell surface or degradation. Although the internalization leads to near-term desensitization to the effects of additional somatostatin exposure; it is also essential to the production to the typical receptor-mediated biologic effect.

With regard to present teaching concerning the ideas and proposed treatments presented within the instant patent, although there are central nervous system interactions between neuronal nitric oxide expression and release and somatostatin pathways, e.g., growth hormone releasing hormone (GHRH) increases secretion of somatostatin and increases somatostatin mRNA levels in the rat paraventricular nucleus via a nitric oxide activation of guanylyl cyclase (Aguila, 1994), the concept that somatostatin receptor agonists interrupt or otherwise modulate peripheral nitric oxide-based signaling has not been previously recognized. Moreover, the issue of the presence or absence of Sst-2A receptors within gastrointestinal muscle is also unresolved; with Murthy, et al., reporting only Sst-3 receptors in isolated intestinal smooth muscle cells, and Reubi, et al., reporting mucosal, neuronal and myenteric plexus staining for Sst-2A receptors in the human intestine, but no apparent staining for Sst-2A receptors within the smooth gastrointestinal muscle itself of humans (Murthy et al., 1996; Reubi et al., 1999). The study by Reubi et al did not examine human esophageal tissues. Although the data from Sternini et al., in the rat gastrointestinal tract are largely in accord with those reported by Reubi, et al, Sternini's group also demonstrated the presence of Sst-2A receptors in the interstitial cells of Cajal in the rat gastrointestinal tract (Sternini et al., 1997). The interstitial cells of Cajal are specialized cell found within the smooth muscles of the gastrointestinal tract, particularly within myenteric plexi, which serve both as a GI motility pacemaker and as a mediator of neurotransmission to the gastrointestinal smooth muscle (Ward et al., 1998; Ward, 2000; Ward et al., 2000; Vanderwinden et al., 1999). Based upon detailed electron-microscopic studies on the human esophagus Faussone-Pellegrini and Cortesini noted, “The ICC are present wherever the esophageal musculature contains smooth muscle cells, but the amount of ICC varies with the esophageal level: in fact, the ICC are abundant in the esophageal body, are even more numerous in the lower esophageal sphincter (LES) and very rare in the gastric (distal) portion of the LES.” (Faussone-Pellegrini and Cortesini, 1985). By a mechanism that is discussed in more detail below, Applicant has discovered that that Sst-2A receptor agonists can and do inhibit nitrergic-based signaling within the gastrointestinal tract, and particularly within the esophagus, lower esophageal sphincter, and sphincter of Oddi.

Somatostatin receptor agonists are used for treatment of neoplastic disorders involving abnormal neuropeptide secretion including acromegaly, carcinoids, and VIPoma; they also are used to treat a variety of forms of non-infections diarrhea, including chemotherapy-induced diarrhea. They also are used in the post-surgical management of a variety of pancreatic diseases, dumping syndrome, intestinal fistulae, and acute gastrointestinal bleeding, especially variceal bleeding. Patients with acromegaly under therapy with octreotide and lanreotide commonly show improvement in acromegaly-associated obstructive sleep apnea and in acromegaly-associated cardiomyopathy. Somatostatin agonists also demonstrate broad anti-inflammatory effects, with some evidence of activity in eosinophilic gastroenteritis (Karalis et al., 1994; Rausch et al., 1997).

Somatostatin receptor agonists useful in the practice of the invention, can be, for example, octreotide acetate and lanreotide acetate, which may be admistered in a variety of methods and dosages which would be known to one of skill in the art.

For example, therapy with injectable peptidic somatostatin receptor (SstR) agonists can begin with subcutaneous administration on a three times daily basis, and then, when acceptable tolerance of the drug is demonstrated, proceed to an intramuscular depot preparation of the peptidic SstR agonist. Therapy with orally bioavailable SstR agonists and for non-peptidic SstR agonists would be based upon their intrinsic molecular potency, the extent of absorption and the rapidity of degradation and or excretion. In the general case it can be assumed that the active dosage range for intravenous administration would range from 0.001 to 5 mg/kg/day, and that the active dosage range for oral administration would range from 0.1 to 50 mg/kg/day. The precise determination of initial and maintenance dosing would be determined by clinical assessment and would be well within the ability of one of skill in the art, without undue experimentation.

An object of the invention can therefore be to provide novel methods of treatment, including novel compositions, and methods for preparing such compositions, for the prevention and/or treatment of gastroesophageal reflux disease (GERD), asthma-associated gastroesophageal reflux (GER), GER-associated asthma, asthma, and related disorders, including, but not limited to, obstructive sleep apnea (OSA), and OSA-associated conditions, including GER, asthma, cardiomyopathy, cardioarrhythmia, congestive heart failure, median nerve compression neuropathy (carpal tunnel syndrome) and cognitive impairment; as well as sleep apnea-associated sudden infant death syndrome (SIDS), central sleep apnea (CSA), as well as CSA-associated conditions, including GER, cardiomyopathy, cardioarrhythmia, congestive heart failure, and cognitive impairment; mixed pattern sleep apneas, including but not limited to post-vascular occlusion sleep apnea, dementia-associated sleep apnea, and alcoholism-related sleep apnea; and excess calpain-activation disorders in tissues where the injured cell population expresses somatostatin receptors; including, but not limited to the central nervous system, heart, liver, kidney, and gastrointestinal tract.

It has surprisingly been found that agonists of somatostatin receptor ligands, including somatostatin receptor agonists of somatostatin receptor types 2 and 5, are particularly useful in the treatment of gastroesophageal reflux disease (GERD), asthma-associated gastroesophageal reflux (GER), GER-associated asthma, asthma, and the related disorders as set forth above.

Since GERD is associated with an abnormally low basal lower esophageal sphincter pressure (LESP), and the degree to which the lower esophageal sphincter pressure departs from the normal range correlates with the severity of GERD and associated conditions, such as Barrett's esophagus, an object of the invention is to provide for a treatment, which increases lower esophageal sphincter pressure.

Since GERD is associated with dysmotility within the esophageal body, particularly in the advanced stages and in patients with Barrett's esophagus, an object of the invention is to provide a treatment, which increases intraesophageal body pressure and esophageal peristaltic motility.

Since esophageal injury from GERD is dependent upon the presence of gastric acid, which is secreted in the stomach and subsequently refluxed into the esophagus, a further object of the invention is to provide for a treatment utilizing agonists of the somatostatin receptors, which reduce the esophageal exposure to acid.

Esophageal injury from GERD is dependent upon the presence within the refluxed gastric contents of the proteolytic enzyme pepsin, which is secreted as pepsinogen by the gastric chief cells in the stomach under stimulation by acetylcholine, gastrin, and cholecystokinin. The present invention therefore, has as yet another object, the inhibition of pepsinogen secretion and its activation to pepsin.

The repeated reflux of duodenal contents and especially, of bile salts into the esophagus in the course of duodenal-gastroesophageal reflux is particularly damaging to the esophageal mucosa. Extensive exposure to bile contents is characteristic of advanced GERD complicated by Barrett's esophagus and early adenocarcinoma occurring in Barrett's esophagus. An object of the invention is therefore to provide a treatment which decreases the rate of entry of bile, bile salts and proteolytic enzymes into the duodenum, thereby decreasing the quantity of bile salts and proteolytic enzymes present within the duodenal contents, thereby decreasing the severity of injury to esophageal mucosa that would arise in the course of duodenogastroesophageal reflux events in GERD.

Patients with GERD experience an increased frequency of transient lower esophageal sphincter relaxation (TLESR) events following ingestion of a meal; this process is triggered by a rise circulating cholecystokinin, stimulated by gastric distension and the entry of lipids into the duodenum. Therefore, another object of the invention is to provide for a treatment which decreases the rate of secretion of cholecystokinin following ingestion of a meal, and to block the peripheral actions of cholecystokinin.

Since GERD is associated with an excess of non-adrenergic, non-cholinergic (NANC) inhibitory signals, which produce the observed low basal lower esophageal sphincter pressure, impaired esophageal motility and increased frequency of transient lower esophageal sphincter relaxation events. The invention therefore has as an object the provision of a treatment which decreases the inhibitory signals to the gut.

There is a striking parallel between the relaxant effects of cholecystokinin (CCK) on the lower esophageal sphincter (LES) and the sphincter of Oddi, which controls the rate of entry of bile into the dudenum. An object of the invention therefore is to provide a treatment which produces parallel constrictive effects on the LES and the sphincter of Oddi.

As discussed above, asthma is associated with a hyperreactive airway, and among the characteristics of a hyperreactive airway is the increased expression of inducible nitric oxide synthase (NOS). An object of the invention therefore is to provide a treatment which inhibits the synthesis of TNF-alpha, IL-1 beta, and INF-gamma by monocytes and T-cell lymphocytes. These cell varieties are characteristically increased in the hyperactive bronchi of asthmatic patients.

Eosinophilic infiltration is characteristically present in the hypersensitive airway tissues of asthmatic patients. It is known that that the normal function of the nuclear transcription factors NF-kappaB and c-fos/AP-1 is necessary for the control of IL-5 and eotaxin genes that are essential for the differentiation, maturation and trafficking of eosinophils, and similarly that the presence and function of the p50 subunit of NF-kappaB is essential to the eosinophilic response to a regional allergic stimulus.

The invention therefore can have as an object, a treatment which inhibits the activation of the nuclear transcription factors NF-kappaB and c-fos/AP-1.

As discussed above, hypoxia occurring during sleep produces an increase in symptoms of both GERD and asthma in the affected patients; the increased asthmatic symptoms may be associated with a loss of effectiveness of the patient's chronic therapy with inhaled glucocorticoids.

The invention therefore can have as an object, a treatment that will, to some degree, protect the patient against the adverse consequences of nocturnal hypoxic episodes, and decrease the severity of those episodes as well.

### DETAILED DESCRIPTION OF THE INVENTION

As discussed above, GERD is associated with an abnormally low basal lower esophageal sphincter pressure (LESP), and the degree to which the lower esophageal sphincter pressure departs from the normal range correlates with the severity of GERD and associated conditions, such as Barrett's esophagus (Coenraad et al., 1998; Lidums and Holloway, 1997; Loughney et al., 1998; Oberg et al., 1999; Singh et al., 1994).

While the effects of somatostatin and somatostatin analogues, such as octreotide and related compounds on esophageal pressure has been explored, there has been no recognition of the use of these compounds in the treatment of GERD and related conditions, prior to Applicant's invention. (Barrioz et al., 1998; Gunshefki et al, 1992). Similarly, while others have explored the use of somatostatin and somatostatin analogues in cases of upper gastrointestinal hemorrhage and in surgical conditions of the pancreas, there has been no recognition of the utility of these compounds in the treatment of GERD and related conditions. (Scarpignato et al., 2001; Scarpignato et al., 1999).

Others, while confirming the published effects of somatostatin in normal humans on esophageal body pressure and post-prandial LESP, found no effect of somatostatin upon on basal esophageal LESP or on the frequency of post-prandial TLESP relaxation events (Straathof et al., 2000). These findings are consistent with the idea that somatostatin receptor agonists primary activity is to decrease excessive neuronal tone that has been mediated through G protein-linked mechanisms. The concept of the instant invention is that GERD is associated with a chronic increase in NANC inhibitory neuronal control, mediated in part by PACAP and VIP stimulated increase in nitric oxide release. Under basal conditions in normal individuals the esophageal LESP is not influenced by an excess of inhibitory neuronal stimuli. The invention therefore is directed to a treatment utilizing agonists of the somatostatin receptors, which increases lower esophageal sphincter pressure that has been lowered by a chronic upregulation of NANC inhibitory tone.

Since GERD is associated with dysmotility within the esophageal body, particularly in the advanced stages and in patients with Barrett's esophagus(Coenraad et al., 1998; DeMeester and DeMeester, 1999; Lidums and Holloway, 1997; Mason and Bremner, 1993), the invention provides a treatment which increases intraesophageal body pressure and motility.

Esophageal injury from GERD is dependent upon the presence of gastric acid, which is secreted in the stomach and subsequently refluxed into the esophagus. Therefore, in one embodiment, the invention provides a treatment utilizing agonists of the somatostatin receptors, which reduce the esophageal exposure to acid.

The inhibition of secretion of gastric acid, by the administration of histamine-2 receptor antagonists, or proton-pump inhibitors, provides the basis for current therapy of GERD. However, those treatments are associated with elevations of serum gastrin levels and atrophy of the gastric mucosa. It is widely known that somatostatin plays a physiologic role in the normal homeostatic control of acid secretion; its intragastric secretion is triggered by lumenal acid exposure of antral D cells, and circulating gastrin exposure of fundal D cells, and inhibited by alkalinity. Through effects on Sstr-2A receptors, somatostatin inhibits the secretion of both gastrin and histamine, the principal triggers for acid secretion by the gastric parietal cells. However, because the acid secretion inhibitory effects of exogenously administered somatostatin analogues are not as profound or as persistent as those attainable by either proton-pump inhibitors or histamine-2 receptor antagonists, they were not considered to be effective for treating duodenal ulcers, and have not been considered candidates for the treatment of GERD.

Acute and chronic therapy with somatostatin receptor agonists, e.g., SstR-2A agonists, inhibits gastric acid secretion without producing elevations in circulating levels of gastrin (Bauer et al., 1989; Karnes et al., 1989, Wyatt et al., 1996). Although this effect may not be competitive with proton pump inhibitors in magnitude and convenience for the treatment of gastric and duodenal ulcers, when these effects are coupled with the promotility and increased LESP effects of SstR-2A agonists on the esophagus, SstR-2A agonists effectively reduce the esophageal exposure to acid.

Esophageal injury from GERD is dependent upon the presence within the refluxed gastric contents of the proteolytic enzyme pepsin, which is secreted as pepsinogen by the gastric chief cells in the stomach under stimulation by acetylcholine, gastrin, and cholecystokinin. The present invention therefore, in another embodiment, inhibits pepsinogen secretion and its activation to pepsin.

The repeated reflux of duodenal contents and especially, of bile acids into the esophagus in the course of duodenogastroesophageal reflux is particularly damaging to the esophageal mucosa and that extensive exposure to bile contents is characteristic of advanced GERD complicated by Barrett's esophagus and early adenocarcinoma occurring in Barrett's esophagus (Stein et al., 1998; Vaezi and Richter, 1995).

The invention therefore provides a treatment with somatostatin receptor agonists which decreases the rate of entry of bile, bile salts and proteolytic enzymes into the duodenum, thereby decreasing the quantity of bile acids and proteolytic enzymes present within the duodenal contents, thereby decreasing the severity of injury to esophageal mucosa that would arise in the course of duodenogastroesophageal reflux events in GERD. While the effect of somatostatin and octreotide on bile flow has been explored, there has been no recognition of their relevance to the pathophysiology of GERD (Gyr and Meier, 1993; Velosy et al., 1999).

Patients with GERD experience an increased frequency of transient lower esophageal sphincter relaxation (TLESR) events following ingestion of a meal.(Dent, 1997; Dent et al., 1988) It is known that endogenous cholecystokinin (CCK) triggers an increase in TLESRs following a meal, and the work of Boulant indicates that this CCK effect is mediated through local release of nitric oxide (Boeckxstaens et al., 1998; Boulant et al., 1994; Clave et al., 1998; Zerbib et al., 1998). While octreotide and related compounds have been recognized as having an impact on circulating lebels of cholecystokinin, there has been no recognition of their utility in the treatment of GERD and related conditions. (Ewins et al., 1992); Shiratori et al., 1991; Stolk et al., 1993; van Berge Henegouwen et al., 1997).

Therefore, yet another embodiment of the invention provides a treatment which utilizes somatostatin receptor agonists to decrease the rate of secretion of cholecystokinin following a meal, and to block the peripheral actions of cholecystokinin.

GERD is associated with an excess of non-adrenergic, non-cholinergic (NANC) inhibitory signals, which produce the observed low basal lower esophageal sphincter pressure; therefore, impaired esophageal motility and increased frequency of transient lower esophageal sphincter relaxation events.

It is known that the interactive signaling molecules vasoactive intestinal peptide (VIP), pituitary adenylate synthase activating peptide (PACAP) and nitric oxide are major NANC inhibitory neurotransmitter substances, which relax smooth muscle within the gastrointestinal tract. Of these substances, the data are strongest for continued participation of nitric oxide in all of the motility events within the esophagus (Boulant et al., 1994; Konturek et al., 1997; Luiking et al., 1998; Murray et al., 1995; Singaram et al., 1994). The work of Aggestrup, Singaram, and others, teaches that vasoactive intestinal peptide (VIP) and nitric oxide are present in significant quantities in the esophagus, and can contribute to gastroesophageal reflux (Aggestrup et al., 1985; Konturek et al., 1997; Luiking et al., 1998; Murray et al., 1995; Ny et al., 1995; Singaram et al., 1994; Uddman et al., 1991).

The high levels of nitric oxide in GERD patients may come from two pathways. The work of Murthy and others teaches that PACAP and VIP produce gastrointestinal smooth muscle relaxation by actions at two separate sites. In studies on rodent stomach and intestine, and intestinal myenteric ganglia, preganglionic release of nitric oxide (from neuronal nitric oxide synthase, nNOS) enhances the release of VIP from post-ganglionic peptidergic neurons (Grider et al., 1992 and 1993). Although comparable studies have not been performed within the esophagus, within stomach and intestinal smooth muscle cells PACAP and VIP activate adenylate cyclase through PAC-1 and VIP-2 receptors, and, by activating the natriuretic peptide clearance receptor, PACAP and VIP trigger Ca++ influx via nifedipine-sensitive calcium channels; the increased intracellular Ca++ activates membrane-bound endothelial nitric oxide synthase (eNOS), increased nitric oxide activates soluble guanylate cyclase, and cGMP-mediated muscle relaxation (Murthy and Makhlouf, 1994; Murthy et al., 1998). There is a similar myogenic nitric oxide synthase in canine lower esophageal sphincer smooth muscle cells, activated by enteric nerve stimulus or by exogenous NO (Salapatek et al., 1998 and 1998). The LES myogenic NOS activation mechanism is known to involve L-type calcium current and K+ channel interactions; however, the specific NOS species has not been identified.

In addition, the intestinalized epithelium in patients with Barrett's esophagus shows high levels of inducible nitric oxide synthase (Wilson et al., 1998). Nitric oxide generated within that epithelium could supplement VIP-induced nitric oxide in producing relaxant effects upon esophageal smooth muscle. In asthma patients with GER, the inhibitory effects of PACAP and VIP can be supplemented by disease-associated nocturnal peripheral release of TNF-alpha and IL-1 beta which can increase expression of inducible nitric oxide synthase in the esophagus.

While others have explored the activity of SsR-2A agonists on VIP and PACAP(Katz and Erstad, 1989; Zindel, 1989; Zeng et al., 1999; Athmann et al, 2000) and their effect on the production of inducible nitric oxide (Chao et al., 1997; Chao et al., 1999), there has been no recognition of their effect on GERD or related conditions.

A further embodiment of the invention therefore provides a treatment with somatostatin receptor agonists which decreases inhibitory signals to the gut by decreasing the rate of secretion of PACAP and VIP both from peptidergic neurons and from post-ganglionic PACAP and VIP neurons that have been activated by a pre-ganglionic nitrergic stimulus produced by release of NO. from neuronal NOS (NOS). These effects are complemented by blocking the peripheral actions of PACAP and VIP, through inhibition of adenylate cyclase activation, and by inhibiting calcium influx via calcium channels in the interstitial cells of Cajal which serve an intermediary function within the esophageal musclulature. Furthermore, somatostatin receptor agonists inhibit the production of inducible nitric oxide through inhibition of the cellular release and peripheral actions of interferon-gamma, TNF-alpha and IL-1 beta. This treatment concept of inhibition of LES relaxation by inhibiting the sequence of nNOS→myogenic NOS activation is consistent with the observation that nNOS deleted (nNOS −/−) mice have an hypertensive (achalasic) lower esophageal sphincter (Kim et al., 1999 and Sivarao et al., 2001).

There is a striking parallel between the effects of cholecystokinin (CCK) on the lower esophageal sphincter (LES) and the sphincter of Oddi, which controls the rate of entry of bile into the dudenum. CCK produces repeated episodes of sphincteric relaxation in both the LES and the sphincter of Oddi (Boeckxstaens et al., 1998; Boulant et al., 1994; Clave et al., 1998; Middelfart et al., 1999; Richards et al., 1993; Shima et al., 1998; Shima et al., 2000; Tokunaga et al., 1993; Zerbib et al., 1998). The effects of CCK on both the LES and the sphincter of Oddi are mediated by release of nitric oxide, presumably from nitrergic neurons (Boulant et al., 1994; Shaffer, 2000; Shima et al., 2000). While the impact of SstR-2A agonists on the LES and the sphincter of Oddi has been explored, there has been no recognition of their utility in the the treatment of GERD and related conditions (Barrioz et al., 1998; Binmoeller et al., 1992; DiFrancesco et al, 1996; Gunshefski et al., 1992; Velosy et al., 1999).

In yet another embodiment, the invention provides a treatment which utilizes somatostatin receptor agonists to produce parallel constrictive effects on the LES and the sphincter of Oddi.

Applicant's treatment with somatostatin receptor agonists addresses the core pathophysiologic basis for the esophageal dysmotility and slack lower esophagel sphincter characteristically present in GERD patients, which exists because of high rates of secretion of PACAP and VIP, leading to increased levels of nitric oxide within the diseased esophageal tissues. The neurotransmitters reinforce each other's production and biologic effect. Therapy with somatostatin receptor agonists decreases inhibitory signals to the gut by decreasing the rates of secretion of PACAP and VIP, by blocking the peripheral actions of PACAP and VIP, through down-regulation of adenylate cyclase, and by inhibiting calcium influx via calcium channels that is the basis of the activation of eNOS within the gastrointestinal smooth muscle (Glassmeier et al., 1998). Furthermore, by reason of their inhibitory effects on the cellular release and peripheral actions of interferon-gamma, TNF-alpha, and IL-1 beta, delivered to the esophagus either by neuronal secretion or as part of the inflammatory response to refluxed gastric content, somatostatin receptor agonists inhibit the production of inducible nitric oxide.

The total effect of therapy is to improve cephalocaudal esophageal motility, provide a tighter lower esophageal sphincter, and a tighter Spinchter of Oddi, reducing the entry of bile into the duodenum. Within this context, the inhibitory effects of somatostatin receptor agonists in reducing the secretion of acid, pepsinogen, histamine, and bile salts, have an increased biologic impact.

In yet another embodiment, the Invention provides a treatment with somatostatin receptor agonists which enhances the utility and decreases the toxicity of serotonin agonists and other prokinetic agents in patients with GERD. Although these agents have useful effects in at least a proportion of GERD patients, the utility of the treatment approach as a whole has been limited by the requirement to use high drug dosages which produce toxicity in a proportion of individuals. Since, as disclosed in the present invention, GERD is a disorder characterized by high levels of inhibitory tone, high doses of the prokinetic agents were needed. With the removal of the inhibitory tone by treatment with somatostatin agonists, the effects of the prokinetic agents, including serotonin agonists, is greatly improved. Confirmation of this concept has recently been published with regard to the effects erythromycin on gastric emptying when combined with octreotide in healthy subjects (Athanasakis, et al., 2002).

Somatostatin 2A receptor agonist therapy as a single treatment program, or in pharmacologically coherent combinations also ameliorates the symptoms of Asthma-associated GER, and GER-associated asthma by reducing the frequency and severity of GER episodes in asthmatics, thereby reducing the frequency and severity of: 1) esophageal acid induced bronchoconstriction by way of a vagal-esophageal-bronchial reflex, 2) heightened bronchial reactivity, 3) microaspiration into the upper airway of refluxed esophageal contents. Moreover, by effects detailed herein, treatment with somatostatin receptor agonists, produces therapeutic effects upon asthmatic conditions, that are independent of these agents effects upon GER.

As discussed above, asthma is associated with a hyper-reactive airway, and among the characteristics of a hyper-reactive airway is the increased expression of inducible nitric oxide synthase (NOS). Untreated asthmatics have an increased content of nitric oxide in their exhaled air. It is known that the expression of inducible nitric oxide synthase (NOS) can be increased in airway epithelial cells by the pro-inflammatory cytokines tumor necrosis factor alpha (TNF-alpha) and interleukin-1 beta (IL-1 beta), and by interferon gamma (IFN-gamma) (Asano et al., 1994; Guo et al., 1997). Although originally perceived as a deliterious part of the inflammatory response in airway cells, inducible nitric oxide synthase, and the nitric oxide it produces are now believed to be a part of the tissue defense mechansims to detoxify reactive oxygen species (ROS), notably superoxide, produced by inflammatory cells (Dweik et al., 2001). In asthmatic patients the inflammatory process to respiratory allergens or other physicochemical stimuli becomes sufficiently severe that the process itself damages lung tissue. Inflammation, with associated ROS release, is amplified in considerable measure by cytokines such as TNF-alpha and IL-1 beta produced locally by lymphocytes and macrophages, and possibly delivered through TNF and IL-1 containing nerves.

In one embodiment, therefore, the invention provides a treatment using somatostatin receptor agonists to inhibit the synthesis and release and block the peripheral actions of TNF-alpha, IL-1 beta, and INF-gamma by monocytes and T-cell lymphocytes, cell varieties are characteristically increased in the hyperactive bronchi of asthmatic patients, as well as within the central nervous system and peripheral nerves.

Eosinophilic infiltration is characteristically present in the hypersensitive airway tissues of asthmatic patients. It is known that that the normal function of the nuclear transcription factors NF-kappaB and c-fos/AP-1 is necessary for the control of IL-5 and eotaxin genes that are essential for the differentiation, maturation and trafficking of eosinophils, and similarly that the presence and function of the p50 subunit of NF-kappaB is essential to the eosinophilic response to a regional allergic stimulus (Hein et al., 1997; Yang et al., 1998).

The invention utilizes somatostatin receptor agonists to inhibit the activation of NF-kappaB and c-fos/AP-1 nuclear transcription factors. Applicant believes that at least in part, this effect may be due the inhibitory actions of SstR-2A agonists upon synthesis and release of the inflammatory cytokine stimulants for NF-kappaB and c-fos/AP-1, as well as the the ability of SstR2A agonists to activate phosphotyrosine protein phosphatases, counterbalancing the initial stimulatory effects of cytokine-activated protein tyrosine kinases (See Todisco et al., 1994; Todisco et al., 1995; Yamashita et al., 1999).

The inventive treatment utilizing somatostatin receptor agonists in the treatment of asthma, provides effects which are part of a broad immunomodulatory action of the somatostatin agonists, which is described in the instant application in detail.

Applicant's invention thus provides broad anti-inflammatory activity, by inhibiting activation of the nuclear transcription factors NF-kappaB and c-fos/AP-1, within monocytes and lymphocytes, thereby inhibiting gene transcription for TNF-alpha, IL-1b, IFN-gamma, and iNOS, and blocking the peripheral effects of ET-1 and substance P. The inhibitory effect of somatostatin agonist therapy on the activation of NF-kappaB and c-fos/AP-1 and the binding of AP-1 resembles a similar inhibitory effect produced by glucocorticoid hormones, which are widely used in the treatment of asthma and other inflammatory disorders.

As discussed herein within the section entitled “Pathophysiologic Context of the Invention”, GERD, GER-associated asthma, asthma-associated GER, and asthma are disorders with a decided diurnal pattern, wherein the symptoms are commonly more severe at night. Within that context, the applicant has discovered that the increased nocturnal severity of symptoms is produced by the diurnal pattern of secretion, within the brain and the peripheral tissues, of particular neuropeptides that have both a somnolent effect and peripheral effects. In GERD patients without asthma, PACAP and VIP would be plausible effector agents, in symptomatic asthma patients TNF-alpha and IL-1 beta would be plausible additional neuropeptides producing the observed effects. Because these patients commonly have disordered sleep, with multiple arousals, they are commonly drowsy during the day as well, plausibly, maintaining elevated neuropeptide secretion(s).

Applicant's invention thus provides broad relief of the hostile diurnal disease pattern by using somatostatin receptor 2A agonists to inhibit excessive secretion of somnolence producing neuropeptides PACAP, VIP, TNF-alpha and IL-1 beta; and by blocking their cellular reactions. Because this therapy relieves symptoms of sleep apnea, it also ameliorates the extent to which apnea-associated hypoxia exacerbates the symptoms of the several disorders.

While others have explored the activity of SsR-2A agonists on sleep function (reviewed in Barkan, 1997 and Krueger et al), prior to the instant invention, there has been no recognition of their effect on GERD, asthma or related conditions.

Since sleep apnea, including obstructive sleep apnea (OSA) and the accompanying hypoxia is widely associated with gastroesophageal reflux in both adults and in infants and young children, including those with a history of apparent life-threatening events (ALTEs), the present invention, therefore, in yet another embodiment, proposes that SstR-2A agonists can be used for the prevention and management of a broad range of OSA-associated illnesses, not limited to these drug's present use in the management of acromegaly-associated OSA. These uses include, but are not limited to, the prevention and management of OSA-associatd GER, OSA-associated cardiomyopathy, OSA-associated cardioarrhythmia, OSA-associated median nerve compression neuropathy (carpal tunnel syndrome), and sleep apnea-associated ALTEs,.

The sleep apnea/hypopnea syndrome, including obstructive sleep apnea, central sleep apnea, with Cheyne-Stokes respiration, and mixed sleep apneas also have major deliterious effects on individuals experiencing them; the data indicate that it is the chronic and recurrent hypoxic periods that produce the injury rather than the particular cause or pattern of apnea (Blackshear et al., 1995; Engleman et al., 2000; Erkinjuntti et al., 1987; Hayakawa et al, 1996; Kimura et al., 1999; Kleopa et al., 1999; Malone et al., 1991; Peled, 1998; Ponikowski et al., 1999; Rosenow, 1994; Stepansky et al., 1997). Since SstR-2A agonists have a broad capacity to inhibit secretion of somnolence-producing neuropeptides, the present invention, in yet another embodiment, proposes that SstR-2A agonists can be applied to the prevention and management of a broad range of sleep apnea- and sleep-hypoventilation-associated illnesses and conditions. These applications include, but are not limited to, the prevention and management of sleep-apnea or sleep-hypoventilation-associated: gastroesophageal reflux, cardiomyopathy, cardioarrhythmia, median nerve compression neuropathy (carpal tunnel syndrome), congestive heart failure, pulmonary hypertension, systemic hypertension, and cognitive impairment. The illnesses or conditions further include apnea or hypoventilation occurring in the context of a present or past cerebrovascular occlusion or hemorrhage, ischemia-reperfusion injury, neuroimmune, neurodegenerative and neuroinflammatory disorders, including amyotrophic lateral sclerosis (ALS), myasthenia gravis, dementia, and alcoholism.

Since tissue injuries, including hypoxic and ischemic injury, are commonly associated with the activation of the calcium-dependent protease calpain and calpain inhibitors lessen the extent of tissue injury (Badalamente et al., 1989; Blomgren, et al., 1999 and 2001; Du, et al., 1999; Edelstein, et al., 1996 and 1999; Harriman, et al., 2000; lizuka, et al., 1991; Iwamoto, et al., 1999; Li, et al., 1996; Shields, et al., 2000; Wang, et al., 2000), and since calpain activation can be inhibited by SstR-2A agonists (Bellocq, et al., 1999), in yet another embodiment, the present invention encompasses the use of SstR-2A agonists to limit damage and amelorate dysfunction in disorders of calpain activation, where the injured cell population expresses somatostatin receptors, these would include but not be limited to injuries produced by ischemia/reperfusion, and other hypoxia, neurodegenerative and neuroinflammatory disorders, dementia, and alcoholism and accidental injury to the central nervous system, heart, liver, kidney, and gastrointestinal tract.

Development of the present invention was stimulated by an event in Applicant's clinical practice of oncology. Octretotide was added to the treatment program of a patient with terminal cancer because the patient's cancer concentrated radiolabelled octreotide on a nuclear medicine scan. Treatment was initiated with subcutaneous octreotide at a dose of 150 microgram twice daily, then three times daily, and then finally the patient was shifted to a longer-acting depot formulation of octreotide (Sandostatin LAR) that was given intramuscularly on a monthly basis. Following the initiation of octreotide administration, the patient reported rapid and complete disappearance of GERD symptoms that had been present for many years. Control of GERD symptomatology was maintained for several months until the patient expired of a common complication of the cancerous condition, unrelated to the therapy.

It is envisioned that therapy with injectable peptidic somatostatin receptor (SstR) agonists can begin with subcutaneous administration on a three times daily basis, and then, when acceptable tolerance of the drug is demonstrated, proceed to an intramuscular depot preparation of the peptidic SstR agonist. Therapy with orally bioavailable SstR agonists and for non-peptidic SstR agonists would be based upon their intrinsic molecular potency, the extent of absorption and the rapidity of degradation and or excretion. In the general case it can be assumed that the active dosage range for intravenous administration would range from 0.001 to 5 mg/kg/day, and that the active dosage range for oral administration would range from 0.1 to 50 mg/kg/day. The precise determination of initial and maintenance dosing would be determined by clinical assessment. The precise determination of what would be considered an effective dose may be based on factors individual to each patient, including their size, age, severity of the condition being treated, and the like. One skilled in the art, specifically a physician, would be able to determine a sufficient amount of active ingredient which would constititute an effective dose without being subjected to undue experimentation.

The following invention shall be further described by the following non-limiting examples are indicative of the treatment approach with formulations that have undergone extensive clinical assessment.

### EXAMPLES

### Example 1

For treatment of GERD, a therapeutically effective amount of octreotide acetate would be from 100 to 600 mcg/day subcutanteously (SC) in two or three divided doses, with the dosage adjusted to patient tolerance and the demonstration of symptomatic benefit, and with objective demonstration of the reduction in reflux events. A rough parallelism is predicted between the dose of octreotide that produces an increase in esophageal motility and esophageal sphincter pressure, and the dose that produces gall bladder distension due to tightening of the sphincter of Oddi. Once control of GERD symptoms is achieved, the dosage can be adjusted to the patient's tolerance, balancing relief of GERD symptoms against the symptoms of loose stools and abdominal discomfort which can be caused by the reduction of bile salts in the intestinal content. Historically, this dosage range can be about 300 mcg/day.

### Example 2

For treatment of GERD, a therapeutically effective amount of octreotide acetate for injectable suspension (depot formulation) Sandostatin LAR® Depot would be from 10 to 30 mg octreotide base equivalent by intra-muscular (IM) injection administered every 4 weeks (28 days). A rough parallelism is predicted between the dose of octreotide that produces an increase in esophageal motility and esophageal sphincter pressure, and the dose that produces gall bladder distension due to tightening of the sphincter of Oddi. Once control of GERD symptoms is achieved, the dosage can be adjusted to the patient's tolerance, balancing relief of GERD symptoms against the symptoms of loose stools and abdominal discomfort which may be caused by the reduction of bile salts in the intestinal content. Extrapolating from the historical experience with octreotide for injection, this dosage range can be about 10 mg/month.

### Example 3

For treatment of GERD, a therapeutically effective amount of lanreotide (Somatuline LA) would be 30 mg administered by IM injection at from 7 to 14 day intervals. A rough parallelism is predicted between the dose of octreotide that produces an increase in esophageal motility and esophageal sphincter pressure, and the dose that produces gall bladder distension due to tightening of the sphincter of Oddi. Once control of GERD symptoms is achieved, the dosage can be adjusted to the patient's tolerance, balancing relief of GERD symptoms against the symptoms of loose stools and abdominal discomfort which may be caused by the reduction of bile salts in the intestinal content. Extrapolating from the historical experience with long-acting lanreotide (Somatuline-LA), this dosage range can be about 30 mg every 14 days.

### Example 4

For treatment of asthma-associated gastroesophageal reflux (GER), a therapeutically effective amount of octreotide acetate would be from 100 to 600 mcg/day subcutanteously (SC) in two or three divided doses, with the dosage adjusted to patient tolerance and the demonstration of symptomatic benefit, and with objective demonstration of the reduction in reflux events. For the GER symptoms a rough parallelism is predicted between the dose of octreotide that produces an increase in esophageal motility and esophageal sphincter pressure, and the dose that produces gall bladder distension due to tightening of the sphincter of Oddi. Once control of GER symptoms is achieved, the dosage can be adjusted to the patient's tolerance, balancing relief of GER symptoms against the symptoms of loose stools and abdominal discomfort which may be caused by the reduction of bile salts in the intestinal content. Historically, this dosage range can be about 300 mcg/day. In those patients where the GER symptoms have arisen in the course of allergic or atopic asthma, that is characterized by diffusely inflamed membranes and a hyperactive airway, stabilization of the airway inflammation by inhaled steroids is indicated before cautiously initiating octreotide therapy at a low dosage of 100 mcg bid, with careful escalation thereafter observing both the GER symptoms and the asthmatic symptoms. When tolerance is assured, a shift to the (depot formulation) Sandostatin LARD® Depot would be appropriate at a low monthly dosage of 10 mg.

### Example 5

For treatment of GER-associated asthma where the asthma symptoms appear to by induced primarily by the GER events, and there is a low suspicion of allergic or atopic asthma, that is characterized by diffusely inflamed membranes and a hyperactive airway, nevertheless, stabilization of the airway inflammation by inhaled steroids is indicated before cautiously initiating octreotide therapy at a low dosage of 100 mcg bid, with careful escalation thereafter observing both the GER symptoms and the asthmatic symptoms. When tolerance is assured, a shift to the (depot formulation) Sandostatin LAR® Depot would be appropriate at a low monthly dosage of 10 mg. If after several months the GER symptoms persist at this dosage level, and asthmatic symptoms are under control, further cautious escalation to 20 mg/month can be explored.

### Example 6

For treatment of asthma the goal of SstR-2A agonist therapy is to reduce the extent and severity of diffuse membrane inflammation and airway hyperactivity, through decreasing the release of the cytokines TNF-alpha, IL-1 beta and interferon gamma, and to block the peripheral effects of these cytokines. These actions complement the anti-inflammatory effects of inhaled glucocorticoids, as does the calpain-inhibitory effect of SstR-2A agonists, which increases glucocorticoid binding and signaling in macrophages (Bellocq, et al., 1999). However, because SstR-2A agonists also reduce neuronal VIP and PACAP release, thereby possibly decreasing airway muscle relaxation, stabilization of the airway inflammation by inhaled steroids is indicated before cautiously initiating octreotide therapy at a low dosage of 100 mcg bid, with careful escalation thereafter with close attention to the acute status of the asthmatic symptoms. When tolerance is assured, a shift to the (depot formulation) Sandostatin LARK® Depot would be appropriate at a low monthly dosage of 10 mg.

### Example 7

For treatment of obstructive sleep apnea (OSA), a therapeutically effective amount of octreotide acetate would be from 100 to 600 mcg/day subcutaneously (SC) in two or three divided doses, with the dosage adjusted to patient tolerance. A rough parallelism is predicted between the dose of octreotide that produces a decrease in sleep apnea events and that which produces gastrointestinal effects on esophageal motility, and pressure changes in the lower esophageal sphincter and the sphincter of Oddi; this can be about 300 mcg/day. When patient tolerance is established, a shift to the (depot formulation) Sandostatin LAR® Depot would be appropriate at 10 to 20 mg/month.

### Example 8

For treatment of sleep apnea/hypopnea-associated GER, a therapeutically effective amount of octreotide acetate would be from 100 to 600 mcg/day subcutaneously (SC) in two or three divided doses, with the dosage adjusted to patient tolerance. A rough parallelism is predicted between the dose of octreotide that produces a decrease in sleep apnea-associated GER and the dose that which produces gall bladder distension through an increase in pressure in the sphincter of Oddi; this range may be about 300 mcg/day. When patient tolerance is established, a shift to the (depot formulation) Sandostatin LAR® Depot would be appropriate at 10 to 20 mg/month. Improvement in the GER symptoms may occur more rapidly than a demonstrable change in sleep apnea events, which may occur over a period of months.

### Example 9

For treatment of sleep apnea-associated asthma there are two goals of SstR-2A agonist therapy; 1) to reduce the extent and severity of diffuse membrane inflammation and airway hyperactivity, and 2) to reduce the severity and frequency of sleep apnea events. Both of these goals are obtained through decreasing the synthesis and release of the cytokines TNF-alpha, IL-1 beta and interferon gamma, and through blocking the peripheral effects of these cytokines. With regard to the airway membranes these actions complement the anti-inflammatory effects of inhaled glucocorticoids, as does the calpain-inhibitory effect of SstR-2A agonists which increases glucocorticoid binding and signaling in macrophages (Bellocq, et al., 1999). However, because SstR-2A agonists also reduce neuronal VIP and PACAP release, thereby possibly decreasing airway muscle relaxation, stabilization of the airway inflammation by inhaled steroids is indicated before cautiously initiating octreotide therapy at a low dosage of 100 mcg bid, with careful escalation thereafter with close attention to the acute status of the asthmatic symptoms. When tolerance is assured, a shift to the (depot formulation) Sandostatin LAR® Depot would be appropriate at a low monthly dosage of 10 mg.

### Example 10

For treatment of sleep apnea-associated cardiomyopathy the goal of SstR-2A agonist therapy is to reduce the severity and frequency of the sleep apnea/hypopnea events, which produce repeated hypoxic occurrences with resulting cardiac damage. A therapeutically effective amount of octreotide acetate would be from 100 to 600 mcg/day subcutaneously (SC) in two or three divided doses, with the dosage adjusted to patient tolerance. A rough parallelism is predicted between the dose of octreotide that produces a decrease in sleep apnea events and that which produces gastrointestinal effects on esophageal motility, and pressure changes in the lower esophageal sphincter and the sphincter of Oddi; this range can be about 300 mcg/day. When patient tolerance is established, a shift to the (depot formulation) Sandostatin LAR® Depot would be appropriate at 10 to 20 mg/month.

### Example 11

For treatment of sleep apnea-associated cardioarrhythmia the goal of SstR-2A agonist therapy is to reduce the severity and frequency of the sleep apnea/hypopnea events, which produce repeated hypoxic occurrences with resulting cardiac damage and cardioarrhythmia. A therapeutically effective amount of octreotide acetate would be from 100 to 600 mcg/day subcutaneously (SC) in two or three divided doses, with the dosage adjusted to patient tolerance. A rough parallelism is predicted between the dose of octreotide that produces a decrease in sleep apnea events and that which produces gastrointestinal effects on esophageal motility, and pressure changes in the lower esophageal sphincter and the sphincter of Oddi; this range may be about 300 mcg/day. When patient tolerance is established, a shift to the (depot formulation) Sandostatin LAR® Depot would be appropriate at 10 to 20 mg/month.

### Example 12

For treatment of sleep apnea/hypopnea that occurs in the context of congestive heart failure the goal of SstR-2A agonist therapy is to reduce the severity and frequency of the sleep apnea/hypopnea events, which produce repeated hypoxic occurrences with resulting cardiac damage. A therapeutically effective amount of octreotide acetate would be from 100 to 600 mcg/day subcutaneously (SC) in two or three divided doses, with the dosage adjusted to patient tolerance. A rough parallelism is predicted between the dose of octreotide that produces a decrease in sleep apnea events and that which produces gastrointestinal effects on esophageal motility, and pressure changes in the lower esophageal sphincter and the sphincter of Oddi; this range may be about 300 mcg/day. When patient tolerance is established, a shift to the (depot formulation) Sandostatin LAR® Depot would be appropriate at 10 to 20 mg/month.

### Example 13

For treatment of sleep apnea/hypopnea that occurs in the context of cognitive impairment and contributes thereto in a variety of geriatric conditions, the goal of SstR-2A agonist therapy is to reduce the severity and frequency of the sleep apnea/hypopnea events, which produce repeated hypoxic occurrences with resulting central nervous system damage. A therapeutically effective amount of octreotide acetate would be from 100 to 600 mcg/day subcutaneously (SC) in two or three divided doses, with the dosage adjusted to patient tolerance. The likely dose required can be about 300 mcg/day. When patient tolerance is established, a shift to the (depot formulation) Sandostatin LAR® Depot would be appropriate at 10 to 20 mg/month.

### Example 14

For treatment of sleep apnea in infants with a history of apparent life-threatening events (ALTE), referred to as a “near-miss” for sudden infant death syndrome (SIDS) the goal of SstR-2A agonist therapy is to reduce the severity and frequency of the sleep apnea/hypopnea events, which produce repeated hypoxic occurrences which may be life-threatening. A therapeutically effective amount of octreotide acetate would be from 2 to 6 micg/kg/day subcutaneously (SC) in two or three divided doses, with the dosage adjusted to patient tolerance. A rough parallelism is predicted between the dose of octreotide that produces a decrease in sleep apnea events and that which produces gastrointestinal effects on small bowel motility (Di Lorenzo, et al. 1998).

### Example 15

For treatment of central sleep apnea (CSA) a therapeutically effective amount of octreotide acetate would be from 100 to 600 mcg/day subcutaneously (SC) in two or three divided doses, with the dosage adjusted to patient tolerance. The expected daily dose can be about 300 mcg/day. When patient tolerance is established, a shift to the (depot formulation) Sandostatin LAR® Depot would be appropriate at 10 to 20 mg/month.

### Example 16

For treatment of mixed pattern sleep apnea a therapeutically effective amount of octreotide acetate would be from 100 to 600 mcg/day subcutaneously (SC) in two or three divided doses, with the dosage adjusted to patient tolerance. The expected daily dose can be about 300 mcg/day. When patient tolerance is established, a shift to the (depot formulation) Sandostatin LAR® Depot would be appropriate at 10 to 20 mg/month.

### Example 17

For treatment of cerebrovascular occlusion-associated sleep apnea a therapeutically effective amount of octreotide acetate would be from 100 to 600 mcg/day subcutaneously (SC) in two or three divided doses, with the dosage adjusted to patient tolerance. The expected daily dose can be about 300 mcg/day. When patient tolerance is established, a shift to the (depot formulation) Sandostatin LAR® Depot would be appropriate at 10 to 20 mg/month.

### Example 18

For treatment of dementia-associated sleep apnea a therapeutically effective amount of octreotide acetate would be from 100 to 600 mcg/day subcutaneously (SC) in two or three divided doses, with the dosage adjusted to patient tolerance. The expected daily dose can be about 300 mcg/day. When patient tolerance is established, a shift to the (depot formulation) Sandostatin LAR® Depot would be appropriate at 10 to 20 mg/month.

### Example 19

For treatment of amyotrophic lateral sclerosis-associated sleep apnea a therapeutically effective amount of octreotide acetate would be from 100 to 600 mcg/day subcutaneously (SC) in two or three divided doses, with the dosage adjusted to patient tolerance. The expected daily dose can be about 300 mcg/day. When patient tolerance is established, a shift to the (depot formulation) Sandostatin LAR® Depot would be appropriate at 10 to 20 mg/month.

### Example 20

For treatment of alcoholism-associated sleep apnea a therapeutically effective amount of octreotide acetate would be from 100 to 600 mcg/day subcutaneously (SC) in two or three divided doses, with the dosage adjusted to patient tolerance. The expected daily dose can be about 300 mcg/day. When patient tolerance is established, a shift to the (depot formulation) Sandostatin LAR® Depot would be appropriate at 10 to 20 mg/month.

### Example 21

For treatment of disorders tissue injury with excess calpain activation a therapeutically effective amount of octreotide acetate would be from 100 to 600 mcg/day subcutaneously (SC) in two or three divided doses, with the dosage adjusted to patient tolerance. The expected daily dose can be about 300 mcg/day. When patient tolerance is established, a shift to the (depot formulation) Sandostatin LARD® Depot would be appropriate at 10 to 20 mg/month.

### Example 22

For the combined use of octreotide and a prokinetic agent in patients with GERD, a therapeutically effective amount of octreotide acetate would be from 100 to 600 mcg/day subcutaneously (SC) in two or three divided doses, with the dosage adjusted to patient tolerance. The expected daily dose can be about 300 mcg/day. When patient tolerance is established, a shift to the (depot formulation) Sandostatin LAR® Depot would be appropriate at 10 mg/month. When stability of the Sandostatin LAR® Depot dosage is established, the prokinetic agent should then be initiated at approximately a quarter of the usual daily dose. The prokinetic agent dosage can then be escalated to meet the symptom goals for the individual patient.

Having thus described in detail preferred embodiments of the present invention, it is to be understood that the invention defined by the appended claims is not to be limited to particular details set forth in the above description, as many apparent variations thereof are possible without departing from the spirit or scope of the present invention.

### DOCUMENTS

Aggestrup, S., Uddman, R., Jensen, S. L., Sundler, F., Schaffalitzky de Muckadell, O., Holst, J. J., Hakanson, R., Ekman, R., and Sorensen, H. R.: Regulatory peptides in the lower esophageal sphincter of man. Regul Pept 10 (2-3): 167-78, 1985.

Aguila, M. C. Growth hormone-releasing factor increases somatostatin release and mRNA levels in the rat periventricular nucleus via nitric oxide by activation of guanylate cyclase, Proc Natl Acad Sci USA. 91: 782-6, 1994.

Ahnaou, A., Basille, M., Gonzalez, B., Vaudry, H., Hamon, M., Adrien, J., and Bourgin, P. Long-term enhancement of REM sleep by the pituitary adenylyl cyclase-activating polypeptide (PACAP) in the pontine reticular formation of the rat, Eur J Neurosci. 11: 4051-8., 1999.

Ahnaou, A., Laporte, A. M., Ballet, S., Escourrou, P., Hamon, M., Adrien, J., and Bourgin, P. Muscarinic and PACAP receptor interactions at pontine level in the rat: significance for REM sleep regulation. Eur J Neurosci 12: 4496-504, 2000.

Arad-Cohen, N., Cohen, A., and Tirosh, E. The relationship between gastroesophageal reflux and apnea in infants. J Pediatr 137:321-6, 2000.

Asano, K., Chee, C. B., Gaston, B., Lilly, C. M., Gerard, C., Drazen, J. M., and Stamler, J. S.: Constitutive and inducible nitric oxide synthase gene expression, regulation, and activity in human lung epithelial cells. Proc Natl Acad Sci USA 91 (21): 10089-93, 1994.

Athanasakis, E., Chrysos, E., Zoras, O. J., Tsiaoussis, J., Karkavatsas, N., and Xynos, E. Octreotide enhances the accelerating effect of erythromycin on gastric emptying in healthy subjects. Aliment Pharmacol 16:1563-70, 2002.

Athmann, C., Zeng, N., Scott, D. R., and Sachs, G. Regulation of parietal cell calcium signaling in gastric glands, Am J Physiol Gastrointest Liver Physiol. 279: G1048-58., 2000.

Badalamente, M. A., Hurst, L. C., and Stracher, A. Neuromuscular recovery using calcium protease inhibition after median nerve repair in primates, Proc Natl Acad Sci USA. 86: 5983-7., 1989

Ballard, R. D. Sleep, respiratory physiology, and nocturnal asthma. Chronobiol Int 16:565-80, 1999.

Barkan, A. Acromegalic arthropathy and sleep apnea, J Endocrinol. 155: S41-4, 1997.

Barnes, P. J., Chung, K. F., and Page, C. P. Inflammatory mediators of asthma: an update, Pharmacol Rev. 50: 515-96, 1998.

Barnes, P. J. Pharmacology of airway smooth muscle, Am J Respir Crit Care Med. 158: S123-32, 1998.

Barrioz, T., Borderie, C., Strock, P., Ingrand, P., Fort, E., Silvain, C., and Beauchant, M.: Effects of octreotide on lower esophageal sphincter in patients with cirrhosis and portal hypertension. Dig Dis Sci 43 (7): 1566-71, 1998.

Bauer, F. E., Hummel, M., Merki, H. S., Schulz, E., Oeder, R., and Marbach, P.: Long-acting somatostatin analog controls acid and gastrin secretion in benign, not in malignant, Zollinger-Ellison syndrome [see comments]. J Clin Gastroenterol 11 (3): 282-6, 1989.

Becker, H. F., Piper, A. J., Flynn, W. E., McNamara, S. G., Grunstein, R. R., Peter, I. H., and Sullivan, C. E. Breathing during sleep in patients with nocturnal desaturation. Am J Respir Crit Care Med. 159:112-8, 1999.

Beglinger, C. and Drewe, J. Somatostatin and octreotide: physiological background and pharmacological application, Digestion. 60: 2-8, 1999.

Bellocq, A., Doublier, S., Suberville, S., Perez, J., Escoubet, B., Fouqueray, B., Puyol, D. R., and Baud, L. Somatostatin increases glucocorticoid binding and signaling in macrophages by blocking the calpain-specific cleavage of Hsp 90, J Biol Chem. 274: 36891-6, 1999.

Binmoeller, K. F., Dumas, R., Harris, A. G., and Delmont, J. P.: Effect of somatostatin analog octreotide on human sphincter of Oddi. Dig Dis Sci 37 (5): 773-7, 1992.

Blackshear, J. L., Kaplan, J., Thompson, R. C., Safford, R. E., and Atkinson, E. J. Nocturnal dyspnea and atrial fibrillation predict Cheyne-Stokes respirations in patients with congestive heart failure, Arch Intern Med. 155: 1297-302., 1995.

Blomgren, K., Hallin, U., Andersson, A. L., Puka-Sundvall, M., Bahr, B. A., McRae, A., Saido, T. C., Kawashima, S., and Hagberg, H. Calpastatin is up-regulated in response to hypoxia and is a suicide substrate to calpain after neonatal cerebral hypoxia-ischemia, J Biol Chem. 274: 14046-52., 1999.

Blomgren, K., Zhu, C., Wang, X., Karlsson, J. O., Leverin, A. L., Bahr, B. A., Mallard, C., and Hagberg, H. Synergistic activation of caspase-3 by m-calpain after neonatal hypoxia-ischemia: a mechanism of “pathological apoptosis”?, J Biol Chem. 276: 10191-8., 2001.

Blum, A. M., Metwali, A., Mathew, R. C., Cook, G., Elliott, D., and Weinstock, J. V.: Granuloma T lymphocytes in murine schistosomiasis mansoni have somatostatin receptors and respond to somatostatin with decreased IFN-gamma secretion. J Immunol 149 (11): 3621-6, 1992.

Boeckxstaens, G. E., Hirsch, D. P., Fakhry, N., Holloway, R. H., D'Amato, M., and Tytgat, G. N.: Involvement of cholecystokininA receptors in transient lower esophageal sphincter relaxations triggered by gastric distension. Am J Gastroenterol 93 (10): 1823-8, 1998.

Boulant, J., Fioramonti, J., Dapoigny, M., Bommelaer, G., and Bueno, L.: Cholecystokinin and nitric oxide in transient lower esophageal sphincter relaxation to gastric distention in dogs [see comments]. Gastroenterology 107 (4): 1059-66, 1994.

Chao, T. C., Chao, H. H., Chen, M. F., and Lin, J. D.: Somatostatin modulates the function of Kupffer cells. Regul Pept 69 (3): 143-9, 1997.

Chao, T. C., Chao, H. H., Lin, J. D., and Chen, M. F.: Somatostatin and octreotide modulate the function of Kupffer cells in liver cirrhosis. Regul Pept 79 (2-3): 117-24, 1999.

Chen, D., Buchanan, G. F., Ding, J. M., Hannibal, J., and Gillette, M. U. Pituitary adenylyl cyclase-activating peptide: A pivotal modulator of glutamatergic regulation of the suprachiasmatic circadian clock. Proc Soc Natl Acad USA. 96:13468-73, 1999.

Chen, Z., Gardi, J, Kushikata, T., Fang, J., and Krueger, J. M. Nuclear factor -kappaB-like activity increases in murine cerebral cortex after sleep deprivation. Am J Physiol 276:R1812-18, 1999.

Chin, K., Ohi, M., Shimizu, K., Nakamura, T., Miyaoka, F., and Mishima, M. Increase in bilirubin levels of patients with obstructive sleep apnea in the morning—a possible explanation of induced heme oxygenase-1, Sleep. 24: 218-23., 2001.

Chowers, Y., Cahalon, L., Lahav, M., Schor, H., Tal, R., Bar-Meir, S., and Levite, M. Somatostatin through its specific receptor inhibits spontaneous and TNF-alpha- and bacteria-induced IL-8 and IL-1 beta secretion from intestinal epithelial cells, J Immunol. 165: 2955-61., 2000.

Clave, P., Gonzalez, A., Moreno, A., Lopez, R., Farre, A., Cusso, X., D'Mato, M., Azpiroz, F., and Lluis, F.: Endogenous cholecystokinin enhances postprandial gastroesophageal reflux in humans through extrasphincteric receptors. Gastroenterology 115 (3): 597-604, 1998.

Coenraad, M., Masclee, A. A., Straathof, J. W., Ganesh, S., Griffloen, G., and Lamers, C. B.: Is Barrett's esophagus characterized by more pronounced acid reflux than severe esophagitis? Am J Gastroenterol 93 (7): 1068-72, 1998.

Csaba, Z. and Dournaud, P. Cellular biology of somatostatin receptors, Neuropeptides. 35: 1-23., 2001.

de Lecea, L., Criado, J. R., Prospero-Garcia, O., Gautvik, K. M., Schweitzer, P., Danielson, P. E., Dunlop, C. L., Siggins, G. R., Henriksen, S. J., and Sutcliffe, J. G. A cortical neuropeptide with neuronal depressant and sleep-modulating properties, Nature. 381: 242-5., 1996.

DeMeester, S. R., and DeMeester, T. R.: The diagnosis and management of Barrett's esophagus. Adv Surg 33: 29-68, 1999.

DeMeester, T. R., Peters, J. H., Bremner, C. G., and Chandrasoma, P. Biology of gastroesophageal reflux disease: pathophysiology relating to medical and surgical treatment, Annu Rev Med. 50: 469-506, 1999.

Dent, J.: Patterns of lower esophageal sphincter function associated with gastroesophageal reflux. Am J Med 103 (5A): 29S-32S, 1997.

Dent, J., Holloway, R. H., Toouli, J., and Dodds, W. J.: Mechanisms of lower oesophageal sphincter incompetence in patients with symptomatic gastrooesophageal reflux. Gut 29 (8): 1020-8, 1988.

Dent, J., Jones, R., Kahrilas, P., and Talley, N. J. Management of gastro-oesophageal reflux disease in general practice, Bmj. 322: 344-7., 2001.

DeVault, K. R. and Castell, D. O. Guidelines for the diagnosis and treatment of gastroesophageal reflux disease. Practice Parameters Committee of the American College of Gastroenterology, Arch Intern Med. 155: 2165-73, 1995.

DeVault, K. R. and Castell, D. O. Updated guidelines for the diagnosis and treatment of gastroesophageal reflux disease. The Practice Parameters Committee of the American College of Gastroenterology, Am J Gastroenterol. 94: 1434-42, 1999.

Di Francesco, V., Angelini, G., Bovo, P., Casarini, M. B., Filippini, M., Vaona, B., Frulloni, L., Rigo, L., Brunori, M. P., and Cavallini, G.: Effect of octreotide on sphincter of Oddi motility in patients with acute recurrent pancreatitis: a manometric study. Dig Dis Sci 41 (12): 2392-6, 1996.

Di Lorenzo, C., Lucanto, C., Flores, A. F., Idries, S., and Hyman, P. E. Effect of octreotide on gastrointestinal motility in children with functional gastrointestinal symptoms, J Pediatr Gastroenterol Nutr. 27: 508-12, 1998.

Dijk, D-J. and Lockley, S. W. Integration of human sleep-wake regulation and circadian rhythmicity. J Appl Physiol 92:852-62, 2002.

Du, S., Rubin, A., Klepper, S., Barrett, C., Kim, Y. C., Rhim, H. W., Lee, E. B., Park, C. W., Markelonis, G. J., and Oh, T. H. Calcium influx and activation of calpain I mediate acute reactive gliosis in injured spinal cord, Exp Neurol. 157: 96-105., 1999.

Dweik, R. A., Comhair, S. A., Gaston, B., Thunnissen, F. B., Farver, C., Thomassen, M. J., Kavuru, M., Hammel, J., Abu-Soud, H. M., and Erzurum, S. C. NO chemical events in the human airway during the immediate and late antigen-induced asthmatic response, Proc Natl Acad Sci USA. 98: 2622-7., 2001.

Dziema, H. And Obrietan, K. PACAP potentiates L-type calcium channel conductance in suprachiasmatic nucleus neurons by activating the MAPK pathway. J Neurophysiol 88:1374-86, 2001.

Edelstein, C. L., Yaqoob, M. M., and Schrier, R. W. The role of the calcium-dependent enzymes nitric oxide synthase and calpain in hypoxia-induced proximal tubule injury, Ren Fail. 18: 501-11., 1996.

Edelstein, C. L., Shi, Y., and Schrier, R. W. Role of caspases in hypoxia-induced necrosis of rat renal proximal tubules, J Am Soc Nephrol. 10: 1940-9., 1999.

Elliott, D. E., Li, J., Blum, A. M., Metwali, A., Patel, Y. C., and Weinstock, J. V.: SSTR2A is the dominant somatostatin receptor subtype expressed by inflammatory cells, is widely expressed and directly regulates T Cell IFN-gamma release. Eur J Immunol 29 (8):2454-63, 1999.

Engleman, H. M., Kingshott, R. N., Martin, S. E., and Douglas, N. J. Cognitive function in the sleep apnea/hypopnea syndrome (SAHS), Sleep. 23: S102-8., 2000.

Erkinjuntti, T., Partinen, M., Sulkava, R., Telakivi, T., Salmi, T., and Tilvis, R. Sleep apnea in multiinfarct dementia and Alzheimer's disease, Sleep. 10: 419-25., 1987.

Ewins, D. L., Javaid, A., Coskeran, P. B., Shah, S., Butler, J., Deprez, P. H., Miell, J., Calam, J., Barrett, J. J., Dawson, J. M., and et al.: Assessment of gall bladder dynamics, cholecystokinin release and the development of gallstones during octreotide therapy for acromegaly. Q J Med 83 (300): 295-306, 1992.

Farup, C., Kleinman, L., Sloan, S., Ganoczy, D., Chee, E., Lee, C., and Revicki, D. The impact of nocturnal symptoms associated with gastroesophageal reflux disease on health-related quality of life. Arch Intern Med 161:45-52.

Faussone-Pellegrini, M. S. and Cortesini, C. Ultrastructural features and localization of the interstitial cells of Cajal in the smooth muscle coat of human esophagus, J Submicrosc Cytol. 17: 187-97, 1985.

Feletou, M., Lonchampt, M., Coge, F., Galizzi, J. P., Bassoullet, C., Merial, C., Robineau, P., Boutin, J. A., Huang, P. L., Vanhoutte, P. M., and Canet, E. Regulation of murine airway responsiveness by endothelial nitric oxide synthase, Am J Physiol Lung Cell Mol Physiol. 281: L258-67., 2001.

Fischer, J. and Jackowski, M. The significance of vasoactive intestinal polypeptide (VIP) in diagnosis of sleep apnea syndrome (English language title), Pneumologie. 43: 584-6., 1989.

Flemons, W. W. Obstructive sleep apnea. N Engl J Med 347: 498-504, 2002.

Fouad, Y. M., Katz, P. O., and Castell, D. O.: Oesophageal motility defects associated with nocturnal gastro-oesophageal reflux on proton pump inhibitors. Aliment Pharmacol Ther 13: 1467-71, 1999.

Frieboes, R. M., Murck, H., Schier, T., Holsboer, F., and Steiger, A. Somatostatin impairs sleep in elderly human subjects, Neuropsychopharmacology. 16: 339-45., 1997.

Galmiche, J. P., Letessier, E., and Scarpignato, C. Treatment of gastro-oesophageal reflux disease in adults, Bmj. 316: 1720-3., 1998.

Georgitis, J. W. The 1997 Asthma Management Guidelines and therapeutic issues relating to the treatment of asthma. National Heart, Lung, and Blood Institute, Chest. 115: 210-7., 1999.

Glassmeier, G., Hopfner, M., Riecken, E. O., Mann, B., Buhr, H., Neuhaus, P., Meyerhof, W., and Scherubl, H.: Inhibition of L-type calcium channels by octreotide in isolated human neuroendocrine tumor cells of the gut. Biochem Biophys Res Commun 250: 511-5, 1998.

Grider, J. R. and Jin, J. G. Vasoactive intestinal peptide release and L-citrulline production from isolated ganglia of the myenteric plexus: evidence for regulation of vasoactive intestinal peptide release by nitric oxide, Neuroscience. 54: 521-6, 1993.

Grider, J. R., Murthy, K. S., Jin, J. G., and Makhlouf, G. M. Stimulation of nitric oxide from muscle cells by VIP: prejunctional enhancement of VIP release, Am J Physiol. 262: G774-8, 1992.

Grunstein, R. R., Ho, K. K., and Sullivan, C. E. Effect of octreotide, a somatostatin analog, on sleep apnea in patients with acromegaly, Ann Intern Med. 121: 478-83., 1994.

Guilleminault, C., Quera-Salva, M. A., Powell, N., Riley, R., Romaker, A., Partinen, M., Baldwin, R., and Nino-Murcia, G. Nocturnal asthma: snoring, small pharynx and nasal CPAP. Eur Respir J 1:902-7, 1988.

Gunshefski, L. A., Rifley, W. J., Slattery, D. W., Schifini, J. J., Hartsuck, M., and Little, A. G.: Somatostatin stimulation of the normal esophagus. Am J Surg 163 (1): 59-62, 1992.

Guo, F. H., Uetani, K., Haque, S. J., Williams, B. R., Dweik, R. A., Thunnissen, F. B., Calhoun, W., and Erzurum, S. C.: Interferon gamma and interleukin 4 stimulate prolonged expression of inducible nitric oxide synthase in human airway epithelium through synthesis of soluble mediators [published erratum appears in J Clin Invest 1997 Sep 1;100(5):1322]. J Clin Invest 100 (4): 829-38, 1997.

Gyr, K. E., and Meier, R.: Pharmacodynamic effects of Sandostatin in the gastrointestinal tract. Digestion 54 (Suppl 1): 14-9, 1993.

Hannibal, J., Ding, J. M., Chen, D., Fahrenkrug, J., Larsen, P. J., Gillette, M. U., and Mikkelsen, J. D. Pituitary adenylate cyclase-activating peptide (PACAP) in the retinohypothalamic tract: A potential daytime regulatory of the biologic clock. J Neurosci 17:2637-44, 1997.

Hannibal, J., Hindersson, P., Knudsen, S. M., Georg, B., and Fahrenkrug, J. The photopigment melanopsin is exclusively present in pituitary adenylate cyclase-activating polypeptide-containing retinal ganglion cells of the retinohypothalamic tract. J Neurosci 22:RC191 (1-7), 2002.

Harding, S. M. Nocturnal asthma: role of nocturnal gastroesophageal reflux. Chronobiol Int 16:641-62, 1999.

Harding, S. M. Gastroesophageal reflux and asthma: insight into the association, J Allergy Clin Immunol. 104: 251-9, 1999.

Harriman, J. F., Waters-Williams, S., Chu, D. L., Powers, J. C., and Schnellmann, R. G. Efficacy of novel calpain inhibitors in preventing renal cell death, J Pharmacol Exp Ther. 294: 1083-7., 2000.

Hayakawa, T., Terashima, M., Kayukawa, Y., Ohta, T., and Okada, T. Changes in cerebral oxygenation and hemodynamics during obstructive sleep apneas, Chest. 109: 916-21., 1996.

Hein, H., Schluter, C., Kulke, R., Christophers, E., Schroder, J. M., and Bartels, J.: Genomic organization, sequence, and transcriptional regulation of the human eotaxin gene. Biochem Biophys Res Commun 237 (3): 537-42, 1997.

Horner, R. L. The neuropharmacology of upper airway motor control in the awake and asleep states: implications for obstructive sleep apnea. Resp Res 2: 286-94, 2001.

Hudgel, D. W. and Thanakitcharu, S. Pharmacologic treatment of sleep-disordered breathing. Am J Respir Crit Care Med 158: 691-9, 1998.

lizuka, K., Kawaguchi, H., and Yasuda, H. Calpain is activated during hypoxic myocardial cell injury, Biochem Med Metab Biol. 46: 427-31., 1991.

Ing, A. J., Ngu, M. C., and Breslin, A. B. Obstructive sleep apnea and gastroesophageal reflux. Am J Med 108 (Suppl 4a):120S-125S, 2000.

Iwamoto, H., Miura, T., Okamura, T., Shirakawa, K., Iwatate, M., Kawamura, S., Tatsuno, H., Ikeda, Y., and Matsuzaki, M. Calpain inhibitor-I reduces infarct size and DNA fragmentation of myocardium in ischemic/reperfused rat heart, J Cardiovasc Pharmacol. 33: 580-6., 1999.

Javaheri, S. A mechanism of central sleep apnea in patients with heart failure. N Engl J Med 341: 949-54, 1999.

Jimenez-Anguiano, A., Garcia-Garcia, F., Mendoza-Ramirez, J. L., Duran- Vazquez, A., and Drucker-Colin, R. Brain distribution of vasoactive intestinal peptide receptors following REM sleep deprivation, Brain Res. 728: 37-46., 1996.

Karalis, K., Mastorakos, G., Chrousos, G. P., and Tolis, G. Somatostatin analogues suppress the inflammatory reaction in vivo, J Clin Invest. 93: 2000-6, 1994.

Karnes, W. E., Maxwell, V., Sytnik, B., Chew, P., and Walsh, J. H.: Prolonged inhibition of meal-stimulated acid secretion and gastrin release following single subcutaneous administration of octreotide (SMS 201-995) in man. Aliment Pharmacol Ther 3 (6): 527-38, 1989.

Katz, M. D., and Erstad, B. L.: Octreotide, a new somatostatin analogue. Clin Pharm 8 (4): 255-73, 1989.

Kim, C. D., Goyal, R. K., and Mashimo, H. Neuronal NOS provides nitrergic inhibitory neurotransmitter in mouse lower esophageal sphincter, Am J Physiol. 277: G280-4, 1999.

Kimura, K., Tachibana, N., Kimura, J., and Shibasaki, H. Sleep-disordered breathing at an early stage of amyotrophic lateral sclerosis, J Neurol Sci. 164: 37-43., 1999.

Kleopa, K. A., Sherman, M., Neal, B., Romano, G. J., and Heiman-Patterson, T. Bipap improves survival and rate of pulmonary function decline in patients with ALS, J Neurol Sci. 164: 82-8., 1999.

Konturek, J. W., Thor, P., Lukaszyk, A., Gabryelewicz, A., Konturek, S. J., and Domschke, W.: Endogenous nitric oxide in the control of esophageal motility in humans. J Physiol Pharmacol 48 (2): 201-9, 1997.

Krieger, J., Sforza, E., Boudewijns, A., Zamagni, M., and Petiau, C. Respiratory effort during obstructive sleep apnea. Role of age and sleep state.

Krueger, J. M., Obal, F., Jr., and Fang, J. Humoral regulation of physiological sleep: cytokines and GHRH, J Sleep Res. 8: 53-9., 1999.

Krueger, J. M., Obal, F., Jr., Fang, J., Kubota, T., and Taishi, P. The role of cytokines in physiological sleep regulation. Ann N Y Acad Sci. 933:211-21, 2001.

Kubin, L., Davies, R. O., and Pack, A. I. Control of uper airway motorneurons during REM sleep. News Physiol Soc. 13: 91-7, 1998.

Kubota, T., Kushikata, T., Fang, J., and Krueger, J. M. Nuclear factor-kappaB inhibitor peptide inhibits spontaneous and interleukin-1beta-induced sleep. Am J Physiol Regul Integr Comp Physiol. 279:R404-13, 2000.

Lamrani, A., Tulliez, M., Chauvelot-Moachon, L., Chaussade, S., Mauprivez, C., Hagnere, A. M., and Vidon, N. Effects of octreotide treatment on early TNF-alpha production and localization in experimental chronic colitis, Aliment Pharmacol Ther. 13: 583-94, 1999.

Lee, P. J., Jiang, B. H., Chin, B. Y., Iyer, N. V., Alam, J., Semenza, G. L., and Choi, A. M. Hypoxia-inducible factor-1 mediates transcriptional activation of the heme oxygenase-1 gene in response to hypoxia, J Biol Chem. 272: 5375-81., 1997.

Leung, R. S. T., and Bradley, T. D. Sleep apnea and cardiovascular disease. Am J Respir Crit Care Med. 164: 2147-65, 2001.

Li, Z., Hogan, E. L., and Banik, N. L. Role of calpain in spinal cord injury: increased calpain immunoreactivity in rat spinal cord after impact trauma, Neurochem Res. 21: 441-8., 1996.

Lidums, I., and Holloway, R.: Motility abnormalities in the columnar-lined esophagus. Gastroenterol Clin North Am 26 (3): 519-31, 1997.

Loughney, T., Maydonovitch, C. L., and Wong, R. K.: Esophageal manometry and ambulatory 24-hour pH monitoring in patients with short and long segment Barrett's esophagus. Am J Gastroenterol 93 (6): 916-9, 1998.

Ludtke, F. E., Maierhof, S., Kohler, H., Bauer, F. E., Tegeler, R., Schauer, A., and Lepsien, G., Helicobacter pylori colonization in surgical patients, Chirurg. 62: 732-8., 1991.

Luiking, Y. C., Weusten, B. L., Portincasa, P., Van Der Meer, R., Smout, A. J., and Akkermans, L. M.: Effects of long-term oral L-arginine on esophageal motility and gallbladder dynamics in healthy humans. Am J Physiol 274 (6 Pt 1): 6984-91, 1998.

Malone, S., Liu, P. P., Holloway, R., Rutherford, R., Xie, A., and Bradley, T. D. Obstructive sleep apnoea in patients with dilated cardiomyopathy: effects of continuous positive airway pressure, Lancet. 338: 1480-4., 1991.

Mason, R. J., and Bremner, C. C.: Motility differences between long-segment and short-segment Barrett's esophagus. Am J Surg 165 (6): 686-9, 1993.

Masuda, A., Shibasaki, T., Kim, Y. S., Imaki, T., Hotta, M., Demura, H., Ling, N., and Shizume, K. The somatostatin analog octreotide inhibits the secretion of growth hormone (GH)-releasing hormone, thyrotropin, and GH in man, J Clin Endocrinol Metab. 69: 906-9., 1989.

McHenry, L., Murthy, K. S., Grider, J. R., and Makhlouf, G. M. Inhibition of muscle cell relaxation by somatostatin: tissue-specific, cAMP-dependent, pertussis toxin-sensitive, Am J Physiol. 261: G45-9., 1991.

Meijer, J. L., Jansen, J. B., Crobach, L. F., Biemond, I., and Lamers, C. B.: Inhibition of omeprazole induced hypergastrinaemia by SMS 201-995, a long acting somatostatin analogue in man. Gut 34 (9): 1186-90, 1993.

Middelfart, H. V., Matzen, P., and Funch-Jensen, P.: Sphincter of Oddi manometry before and after laparoscopic cholecystectomy. Endoscopy 31 (2): 146-51, 1999.

Moller, D. E., Moses, A. C., Jones, K., Thorner, M. O., and Vance, M. L. Octreotide suppresses both growth hormone (GH) and GH-releasing hormone (GHRH) in acromegaly due to ectopic GHRH secretion, J Clin Endocrinol Metab. 68: 499-504., 1989.

Murray, J. A., Ledlow, A., Launspach, J., Evans, D., Loveday, M., and Conklin, J. L.: The effects of recombinant human hemoglobin on esophageal motor functions in humans. Gastroenterology 109 (4): 1241-8, 1995.

Murthy, K. S., Coy, D. H., and Makhlouf, G. M. Somatostatin receptor-mediated signaling in smooth muscle. Activation of phospholipase C-beta3 by Gbetagamma and inhibition of adenylyl cyclase by Galphail and Galphao, J Biol Chem. 271: 23458-63, 1996.

Murthy, K. S., and Makhlouf, G. M.: Vasoactive intestinal peptide/pituitary adenylate cyclase-activating peptidedependent activation of membrane-bound NO synthase in smooth muscle mediated by pertussis toxin-sensitive Gil -2. J Biol Chem 269 (23): 15977-80, 1994.

Murthy, K. S., Teng, B., Jin, J., and Makhlouf, G. M.: G protein-dependent activation of smooth muscle eNOS via natriuretic peptide clearance receptor. Am J Physiol 275 (6 Pt 1): C1409-16, 1998.

Ny, L., Alm, P., Ekstrom, P., Larsson, B., Grundemar, L., and Andersson, K. E. Localization and activity of haem oxygenase and functional effects of carbon monoxide in the feline lower oesophageal sphincter, BrJ Pharmacol. 118: 392-9., 1996.

Ny, L., Grundemar, L., Larsson, B., Alm, P., Ekstrom, P., and Andersson, K. E. Carbon monoxide as a putative messenger molecule in the feline lower oesophageal sphincter of the cat, Neuroreport. 6: 1389-93., 1995.

Ny, L., Larsson, B., Alm, P., Ekstrom, P., Fahrenkrug, J., Hannibal, J., and Andersson, K. E. Distribution and effects of pituitary adenylate cyclase activating peptide in cat and human lower oesophageal sphincter, Br J Pharmacol. 116: 2873-80, 1995.

Oberg, S., DeMeester, T. R., Peters, J. H., Hagen, J. A., Nigro, J. J., DeMeester, S. R., Theisen, J., Campos, G. M., and Crookes, P. F.: The extent of Barrett's esophagus depends on the status of the lower esophageal sphincter and the degree of esophageal acid exposure. J Thorac Cardiovasc Surg 117 (3): 572-80, 1999.

Pando, M. P. and Sassone-Corsi, P. Signaling to the mammalian circadian clocks: in pursuit of the primary mammalian circadian photoreceptor. SCI STKE 2001 (107) RE 16

Patel, Y. C. Somatostatin and its receptor family, Front Neuroendocrinol. 20: 157-98, 1999.

Pedersen, M. E. F., Dorrington, K. L., and Robbins, P. A. Effects of somatostatin on breathing in humans. J Physiol 521: 289-97, 1999.

Peghini, P. L., Katz, P. O., Bracy, N. A., and Castell, D. O. Nocturnal recovery of gastric acid secretion with twice-daily dosing of proton pump inhibitors. Am J Gastroenterol 93:763-7, 1998.

Peled, N., Greenberg, A., Pillar, G., Zinder, O., Levi, N., and Lavie, P. Contributions of hypoxia and respiratory disturbance index to sympathetic activation and blood pressure in obstructive sleep apnea syndrome, Am J Hypertens. 11: 1284-9, 1998.

Peluso, G., Petillo, O., Melone, M. A., Mazzarella, G., Ranieri, M., and Tajana, G. F.: Modulation of cytokine production in activated human monocytes by somatostatin. Neuropeptides 30 (5): 443-51, 1996.

Ponikowski, P., Anker, S. D., Chua, T. P., Francis, D., Banasiak, W., Poole-Wilson, P. A., Coats, A. J., and Piepoli, M. Oscillatory breathing patterns during wakefulness in patients with chronic heart failure: clinical implications and role of augmented peripheral chemosensitivity, Circulation. 100: 2418-24., 1999.

Rausch, T., Gyr, K., Wegmann, W., Germer, M., and Meier, R. Symptomatic therapy of severe diarrhea in eosinophilic gastroenteritis with the somatostatin analog octreotide (Sandostatin) (English language title), Schweiz Med Wochenschr Suppl. 89: 9S-13S, 1997.

Reubi, J. C., Laissue, J. A., Waser, B., Steffen, D. L., Hipkin, R. W., and Schonbrunn, A. Immunohistochemical detection of somatostatin sst2a receptors in the lymphatic, smooth muscular, and peripheral nervous systems of the human gastrointestinal tract: facts and artifacts, J Clin Endocrinol Metab. 84: 2942-50, 1999.

Richards, R. D., Yeaton, P., Shaffer, H. A., Jr., Pambianco, D. J., Pruett, T. L., Stevenson, W. C., Mittal, R. K., and McCallum, R. W.: Human sphincter of Oddi motility and cholecystokinin response following liver transplantation. Dig Dis Sci 38 (3): 462-8, 1993.

Rohrer, S. P., Birzin, E. T., Mosley, R. T., Berk, S. C., Hutchins, S. M., Shen, D. M., Xiong, Y., Hayes, E. C., Parmar, R. M., Foor, F., Mitra, S. W., Degrado, S. J., Shu, M., Klopp, J. M., Cai, S. J., Blake, A., Chan, W. W., Pasternak, A., Yang, L., Patchett, A. A., Smith, R. G., Chapman, K. T., and Schaeffer, J. M. Rapid identification of subtype-selective agonists of the somatostatin receptor through combinatorial chemistry, Science. 282: 737-40., 1998.

Rosenow, F., Reuter, S., Szelies, B., Deuss, U., Hildebrandt, G., Schneider, D., Winkelmann, W., and Heiss, W. D. Sleep apnoea in acromegaly--prevalence, pathogenesis and therapy. Report on two cases, Presse Med. 23: 1203-8., 1994.

Rossiter, A., Guelrud, M., Souney, P. F., Mendoza, S., Rossiter, G., and Gelrud, D. High vasoactive intestinal polypeptide plasma levels in patients with Barrett's esophagus, Scand J Gastroenterol. 26: 572-6, 1991.

Salapatek, A. M., Wang, Y. F., Mao, Y. K., Lam, A., and Daniel, E. E. Myogenic nitric oxide synthase activity in canine lower oesophageal sphincter: morphological and functional evidence, Br J Pharmacol. 123: 1055-64, 1998.

Salapatek, A. M., Wang, Y. F., Mao, Y. K., Mori, M., and Daniel, E. E. Myogenic NOS in canine lower esophageal sphincter: enzyme activation, substrate recycling, and product actions, Am J Physiol. 274: C1145-57, 1998.

Scarpignato, C. and Pelosini, I. Somatostatin for upper gastrointestinal hemorrhage and pancreatic surgery. A review of its pharmacology and safety, Digestion. 60. 1-16, 1999.

Scarpignato, C. and Pelosini, I. Somatostatin analogs for cancer treatment and diagnosis: an overview, Chemotherapy. 47: 1-29., 2001.

See, C. C., Newman, L. J., Berezin, S., Glassman, M. S., Medow, M. S., Dozor, A. J., and Schwarz, S. M. Gastroesophageal reflux-induced hypoxemia in infants with apparent life-threatening event(s). Am J Dis Child 143:951-4, 1989.

Shaffer, E. A.: Review article: control of gall-bladder motor function [In Process Citation]. Aliment Pharmacol Ther 14 (Suppl 2): 2-8, 2000.

Shields, D. C., Schaecher, K. E., Hogan, E. L., and Banik, N. L. Calpain activity and expression increased in activated glial and inflammatory cells in penumbra of spinal cord injury lesion, J Neurosci Res. 61: 146-50., 2000.

Shima, Y., Mori, M., Harano, M., Tsuge, H., Tanaka, N., and Yamazato, T.: Nitric oxide mediates cerulein-induced relaxation of canine sphincter of Oddi. Dig Dis Sci 43 (3): 547-53, 1998.

Shima, Y., Mori, M., Takakura, N., Tanaka, N., Yokoi, I., Kabuto, H., and Yamazato, T.: Continuous monitoring of nitric oxide release induced by cholecystokinin from the choledochal sphincter in guinea pigs. Digestion 61 (2): 135-9, 2000.

Shiratori, K., Watanabe, S., and Takeuchi, T.: Somatostatin analog, SMS 201-995, inhibits pancreatic exocrine secretion and release of secretin and cholecystokinin in rats. Pancreas 6 (1): 23-30, 1991.

Singaram, C., Sengupta, A., Sweet, M. A., Sugarbaker, D. J., and Goyal, R. K.: Nitrinergic and peptidergic innervation of the human oesophagus. Gut 35 (12): 1690-6, 1994.

Singh, P., Taylor, R. H., and Colin-Jones, D. G.: Esophageal motor dysfunction and acid exposure in reflux esophagitis are more severe if Barrett's metaplasia is present. Am J Gastroenterol 89 (3): 349-56, 1994.

Sivarao, D. V., Mashimo, H. L., Thatte, H. S., and Goyal, R. K. Lower Esophageal Sphincter Is Achalasic in nNOS(−/−) and Hypotensive in W/W(v) Mutant Mice, Gastroenterology. 121: 34-42., 2001.

Sontag, S. J. Gastroesophageal reflux disease and asthma, J Clin Gastroenterol. 30: S9-30, 2000.

Spier, A. D. and de Lecea, L. Cortistatin: a member of the somatostatin neuropeptide family with distinct physiological functions, Brain Res Brain Res Rev. 33: 228-41., 2000

Stein, H. J., Kauer, W. K., Feussner, H., and Siewert, J. R.: Bile reflux in benign and malignant Barrett's esophagus: effect of medical acid suppression and nissen fundoplication. J Gastrointest Surg 2 (4): 333-41, 1998.

Stepansky, R., Weber, G., and Zeitlhofer, J. Sleep apnea and cognitive dysfunction in myasthenia gravis, Acta Med Austriaca. 24: 128-31., 1997.

Sternini, C., Wong, H., Wu, S. V., de Giorgio, R., Yang, M., Reeve, J., Jr., Brecha, N. C., and Walsh, J. H. Somatostatin 2A receptor is expressed by enteric neurons, and by interstitial cells of Cajal and enterochromaffin-like cells of the gastrointestinal tract, J Comp Neurol. 386: 396-408, 1997.

Stolk, M. F., van Erpecum, K. J., Koppeschaar, H. P., de Bruin, W. I., Jansen, J. B., Lamers, C. B., and van Berge Henegouwen, G. P.: Postprandial gall bladder motility and hormone release during intermittent and continuous subcutaneous octreotide treatment in acromegaly. Gut 34 (6): 808-13, 1993.

Straathof, J. W., Tieleman, S., Lamers, C. B., and Masclee, A. A. Effect of somatostatin on lower esophageal sphincter characteristics in man, Scand J Gastroenterol. 35: 910-5., 2000.

Sutcliffe, J. G. and de Lecea, L. Novel neurotransmitters for sleep and energy homeostasis, Results Probl Cell Differ. 26: 239-55., 1999.

Syabbalo, N. Chronobiology and chronopathophysiology of nocturnal asthma. Int J Clin Pract 51:455-62, 1997.

Takahashi, S., Kapas, L., Fang, J., and Krueger, J. M. Somnogenic relationships between tumor necrosis factor and interleukin-1, Am J Physiol. 276: R1132-40., 1999.

Thorup, C., Jones, C. L., Gross, S. S., Moore, L. C., and Goligorsky, M. S. Carbon monoxide induces vasodilation and nitric oxide release but suppresses endothelial NOS, Am J Physiol. 277: F882-9., 1999.

Todisco, A., Campbell, V., Dickinson, C. J., DeIValle, J., and Yamada, T.: Molecular basis for somatostatin action: inhibition of c-fos expression and AP-1 binding. Am J Physiol 267 (2 Pt 1): 6245-53, 1994.

Todisco, A., Seva, C., Takeuchi, Y., Dickinson, C. J., and Yamada, T.: Somatostatin inhibits AP-1 function via multiple protein phosphatases. Am J Physiol 269 (1 Pt 1): 6160-6, 1995.

Tokunaga, Y., Cox, K. L., Itasaka, H., Concepcion, W., Nakazato, P., and Esquivel, C. O.: Characterization of cholecystokinin receptors on the human sphincter of Oddi. Surgery 114 (5): 942-50, 1993.

Vaezi, M. F., and Richter, J. E.: Synergism of acid and duodenogastroesophageal reflux in complicated Barrett's esophagus. Surgery 117 (6): 699-704, 1995.

van Berge Henegouwen, M. I., van Gulik, T. M., Akkermans, L. M., Jansen, J. B., and Gouma, D. J.: The effect of octreotide on gastric emptying at a dosage used to prevent complications after pancreatic surgery: a randomised, placebo controlled study in volunteers. Gut 41 (6): 758-62, 1997.

Velosy, B., Madacsy, L., Szepes, A., Pavics, L., Csernay, L., and Lonovics, J.: The effects of somatostatin and octreotide on the human sphincter of Oddi. Eur J Gastroenterol Hepatol 11 (8): 897-901, 1999.

Vanderwinden, J. M. Role of Interstitial Cells of Cajal and their relationship with the enteric nervous system, Eur J Morphol. 37: 250-6, 1999.

Vgontzas, A. N., Papanicolaou, D. A., Bixler, E. O., Kales, A., Tyson, K., and Chrousos, G. P. Elevation of plasma cytokines in disorders of excessive daytime sleepiness: role of sleep disturbance and obesity, J Clin Endocrinol Metab. 82: 1313-6., 1997.

Vgontzas, A. N., Papanicolaou, D. A., Bixler, E. O., Lotsikas, A., Zachman, K., Kales, A., Prolo, P., Wong, M., Licinio, J., Gold, P. W., Hermidia, R., C., Mastorakos, G., and Chrousos, G. P. Circadian interleukin-6 secretion and quantity and depth of sleep. J Clin Endocrinol Metab. 84:2603-7, 1999.

Vgontzas, A. N., Papanicolaou, D. A., Bixler, E. O., Hopper, K., Lotsikas, A., Lin, H. M., Kales, A., and Chrousos, G. P. Sleep apnea and daytime sleepiness and fatigue: relation to visceral obesity, insulin resistance, and hypercytokinemia, J Clin Endocrinol Metab. 85: 1151-8., 2000.

Wang, M. S., Wu, Y., Culver, D. G., and Glass, J. D. Pathogenesis of axonal degeneration: parallels between Wallerian degeneration and vincristine neuropathy, J Neuropathol Exp Neurol. 59: 599-606., 2000.

Ward, S. M., Morris, G., Reese, L., Wang, X. Y., and Sanders, K. M. Interstitial cells of Cajal mediate enteric inhibitory neurotransmission in the lower esophageal and pyloric sphincters, Gastroenterology. 115: 314-29, 1998.

Ward, S. M. Interstitial cells of Cajal in enteric neurotransmission, Gut. 47: iv4O-3; discussion iv52., 2000.

Ward, S. M., Beckett, E. A., Wang, X., Baker, F., Khoyi, M., and Sanders, K. M. Interstitial cells of Cajal mediate cholinergic neurotransmission from enteric motor neurons, J Neurosci. 20: 1393-403, 2000.

Werth, E., Acherman, P., and Borbely, A. A. Selective REM sleep deprivation during daytime II. Muscle atonia in non-REM sleep. Am J Physiol Regul Integr Comp Physiol 283: R527-32, 2002.

Wilson, K. T., Fu, S., Ramanujam,-K. S., and Meltzer, S. J.: Increased expression of inducible nitric oxide synthase and cyclooxygenase-2 in Barrett's esophagus and associated adenocarcinomas. Cancer Res 58 (14): 2929-34, 1998.

Wyatt, M. A., Jarvie, E., Feniuk, W., and Humphrey, P. P.: Somatostatin sst2 receptor-mediated inhibition of parietal cell function in rat isolated gastric mucosa. Br J Pharmacol 119 (5): 905-10, 1996.

Xie, A., Skatrud, J. B., Puleo, D. S., Rahko, P. S., and Dempsey, J. A. Apnea-hypopnea threshold for CO2 in patients with congestive heart failure. Am J Respir Crit Care Med 165: 1245-50, 2002.

Xie, A., Skatrud, J. B., and Dempsey, J. A. Effect of hypoxia on the hypopneic and apneic threshold for CO2 in sleeping humans. J Physiol 535: 269-78, 2001.

Xue, L., Farrugia, G., Miller, S. M., Ferris, C. D., Snyder, S. H., Szurszewski, J. H. Carbon monoxide and nitric oxide as coneurotransmitters in the enteric nervous system: evidence from genomic deletion of biosynthetic enzymes. Proc Natl Acad Sci U S A. 97:1851-5. 2000.

Yamashita, M., Dimayuga, P., Kaul, S., Shah, P. K., Regnstrom, J., Nilsson, J., and Cercek, B.: Phosphatase activity in the arterial wall after balloon injury: effect of somatostatin analog octreotide. Lab Invest 79 (8): 935-44, 1999.

Yang, L., Berk, S. C., Rohrer, S. P., Mosley, R. T., Guo, L., Underwood, D. J., Arison, B. H., Birzin, E. T., Hayes, E. C., Mitra, S. W., Parmar, R. M., Cheng, K., Wu, T. J., Butler, B. S., Foor, F., Pasternak, A., Pan, Y., Silva, M., Freidinger, R. M., Smith, R. G., Chapman, K., Schaeffer, J. M., and Patchett, A. A. Synthesis and biological activities of potent peptidomimetics selective for somatostatin receptor subtype 2, Proc Natl Acad Sci USA. 95: 10836-41, 1998.

Yang, L., Cohn, L., Zhang, D. H., Homer, R., Ray, A., and Ray, P.: Essential role of nuclear factor kappaB in the induction of eosinophilia in allergic airway inflammation. J Exp Med 188 (9): 1739-50, 1998.

Young, T., Peppard, P. E. and Gottlieb, D. J. Epidemiology of obstructive sleep apnea, A population health perspective. Am J Resp Crit Care Med 165: 1217-39, 2001.

Zeng, N., Athmann, C., Kang, T., Walsh, J. H., and Sachs, G. Role of neuropeptide-sensitive L-type Ca(2+) channels in histamine release in gastric enterochromaffin-like cells, Am J Physiol. 277: G1268-80, 1999.

Zerbib, F., Bruley Des Varannes, S., Scarpignato, C., Leray, V., D'Amato, M., Roze, C., and Galmiche, J. P.: Endogenous cholecystokinin in postprandial lower esophageal sphincter function and fundic tone in humans. Am J Physiol 275 (6 Pt 1): 61266-73, 1998.

Zindel, L. R.: Debut of a somatostatin analog: octreotide in review. Conn Med 53 (12): 741-4, 1989.

U.S. PATENT DOCUMENTS

| | | | |
|---|---|---|---|
| 4,087,390 | to Shields | 4,612,366 | to Nutt |
| 4,115,554 | to Veber | 4,621,073 | to Friedrich et al. |
| 4,113,782 | to Vate, Jr. et al. | 4,663,435 | to Brady |
| 4,139,526 | to Veber | 4,728,638 | to Bauer et al. |
| 4,140,767 | to Veber | 4,904,642 | to Coy et al. |
| 4,145,337 | to Daiman et al. | 5,491,131 | to Puyol et al. |
| 4,146,612 | to Veber | 5,506,339 | to Coy et al. |
| 4,209,441 | to Lapidus et al. | 5,569,741 | to Coy et al. |
| 4,210,636 | to Lien et al. | 5,750,499 | to Hoeger et al. |
| 4,235,886 | to Freidinger et al. | 5,968,903 | to Kaneko et al. |
| 4,280,953 | to Guillemin et al. | 6,020,349 | to Ankersen et al. |
| 4,316,891 | to Guillemin et al. | 6,025,372 | to Yang et al. |
| 4,360,516 | to Freidinger et al. | 6,057,338 | to Yang et al. |
| 4,393,050 | to Vale, Jr. et al. | 6,063,796 | to Yang et al. |
| 4,427,661 | to Curley et al. | 6,083,960 | to Ankersen et al. |
| 4,470,974 | to Goldenberg et al. | 6,117,880 | to Guo et al. |
| 4,585,755 | to Morgan et al. | 6,127,343 | to Ankersen et al. |
| 4,611,054 | to Freidinger | | |

What is claimed:

1. A method of preventing or treating gastroesophageal reflux disease (GERD), asthma-associated gastroesophageal reflux (GER), GER-associated asthma and asthma, or related disorders, which method comprises administering an effective amount of a composition comprising a somatostatin receptor agonist to a human patient in need thereof.

2. The method according to claim 1, wherein the somatostatin receptor agonist is selected from the group consisting of agonists of somatostatin receptor types 2 and 5.

3. The method according to claim 1, wherein the somatostatin receptor agonist is selected from the group consisting of agonists of somatostatin type 2A receptor.

4. The method according to claim 1, wherein the somatostatin receptor agonist is selected from the group consisting of non-peptide somatostatin agonists.

5. The method according to claim 1, wherein the somatostatin receptor agonist is octreotide acetate.

6. The method according to claim 1, wherein the somatostatin receptor agonist is lanreotide.

7. The method of preventing or treating gastroesophageal reflux disease (GERD), asthma-associated gastroesophageal reflux (GER), GER-associated asthma and asthma, or related disorders, which method comprises administering a composition comprising an effective amount of a somatostatin receptor agonist sufficient to inhibit pepsinogen secretion and its activation to pepsin to a human patient in need thereof.

8. The method according to claim 7, wherein the somatostatin receptor agonist is selected from the group consisting of agonists of somatostatin receptor types 2 and 5.

9. The method according to claim 7, wherein the somatostatin receptor agonist is selected from the group consisting of agonists of somatostatin type 2A receptor.

10. The method according to claim 7, wherein the somatostatin receptor agonist is selected from the group consisting of non-peptide somatostatin agonists.

11. The method according to claim 7, wherein the somatostatin receptor agonist is octreotide acetate.

12. The method according to claim 7, wherein the somatostatin receptor agonist is lanreotide.

13. The method of preventing or treating gastroesophageal reflux disease (GERD), asthma-associated gastroesophageal reflux (GER), GER-associated asthma and asthma, or related disorders, which method comprises administering a composition comprising an effective amount of a somatostatin receptor agonist sufficient to increase lower esophageal sphincter pressure to a human patient in need thereof.

14. The method according to claim 13, wherein the somatostatin receptor agonist is selected from the group consisting of agonists of somatostatin receptor types 2 and 5.

15. The method according to claim 13, wherein the somatostatin receptor agonist is selected from the group consisting of agonists of somatostatin type 2A receptor.

16. The method according to claim 13, wherein the somatostatin receptor agonist is selected from the group consisting of non-peptide somatostatin agonists.

17. The method according to claim 13, wherein the somatostatin receptor agonist is octreotide acetate.

18. The method according to claim 13, wherein the somatostatin receptor agonist is lanreotide.

19. The method of preventing or treating gastroesophageal reflux disease (GERD), asthma-associated gastroesophageal reflux (GER), GER-associated asthma and asthma, or related disorders, which method comprises administering a composition comprising an effective amount of a somatostatin receptor agonist sufficient to increase intraesophageal body pressure and motility to a human patient in need thereof.

20. The method according to claim 19, wherein the somatostatin receptor agonist is selected from the group consisting of agonists of somatostatin receptor types 2 and 5.

21. The method according to claim 19, wherein the somatostatin receptor agonist is selected from the group consisting of agonists of somatostatin type 2A receptor.

22. The method according to claim 19, wherein the somatostatin receptor agonist is selected from the group consisting of non-peptide somatostatin agonists.

23. The method according to claim 19, wherein the somatostatin receptor agonist is octreotide acetate.

24. The method according to claim 19, wherein the somatostatin receptor agonist is lanreotide.

25. The method of preventing or treating gastroesophageal reflux disease (GERD), asthma-associated gastroesophageal reflux (GER), GER-associated asthma and asthma, or related disorders, which method comprises administering a composition comprising an effective amount of a somatostatin receptor agonist sufficient to reduce esophageal exposure to acid to a human patient in need thereof.

26. The method according to claim 25, wherein the somatostatin receptor agonist is selected from the group consisting of agonists of somatostatin receptor types 2 and 5.

27. The method according to claim 25, wherein the somatostatin receptor agonist is selected from the group consisting of agonists of somatostatin type 2A receptor.

28. The method according to claim 25, wherein the somatostatin receptor agonist is selected from the group consisting of non-peptide somatostatin agonists.

29. The method according to claim 25, wherein the somatostatin receptor agonist is octreotide acetate.

30. The method according to claim 25, wherein the somatostatin receptor agonist is lanreotide.

31. The method of preventing or treating gastroesophageal reflux disease (GERD), asthma-associated gastroesophageal reflux (GER), GER-associated asthma and asthma, or related disorders, which method comprises administering a composition comprising an effective amount of a somatostatin receptor agonist sufficient to decrease the rate of entry of bile, bile salts and proteolytic enzymes into the duodenum, thereby decreasing the quantity of bile acids and proteolytic enzymes present within the duodenal contents to a human patient in need thereof.

32. The method according to claim 31, wherein the somatostatin receptor agonist is selected from the group consisting of agonists of somatostatin receptor types 2 and 5.

33. The method according to claim 31, wherein the somatostatin receptor agonist is selected from the group consisting of agonists of somatostatin type 2A receptor.

34. The method according to claim 31, wherein the somatostatin receptor agonist is selected from the group consisting of non-peptide somatostatin agonists.

35. The method according to claim 31 wherein the somatostatin receptor agonist is octreotide acetate.

36. The method according to claim 31, wherein the somatostatin receptor agonist is lanreotide.

37. The method of preventing or treating gastroesophageal reflux disease (GERD), asthma-associated gastroesophageal reflux (GER), GER-associated asthma and asthma, or related disorders, which method comprises administering a composition comprising an effective amount of a somatostatin receptor agonist sufficient to decrease the rate of secretion of cholecystokinin to a human patient in need thereof.

38. The method according to claim 37, wherein the somatostatin receptor agonist is selected from the group consisting of agonists of somatostatin receptor types 2 and 5.

39. The method according to claim 37, wherein the somatostatin receptor agonist is selected from the group consisting of agonists of somatostatin type 2A receptor.

40. The method according to claim 37, wherein the somatostatin receptor agonist is selected from the group consisting of non-peptide somatostatin agonists.

41. The method according to claim 37, wherein the somatostatin receptor agonist is octreotide acetate.

42. The method according to claim 37, wherein the somatostatin receptor agonist is lanreotide.

43. The method of preventing or treating gastroesophageal reflux disease (GERD), asthma-associated gastroesophageal reflux (GER), GER-associated asthma and asthma, or related disorders, which method comprises administering a composition comprising an effective amount of a somatostatin receptor agonist sufficient to decrease an excessive rate of secretion within the brain and/or the peripheral nervous system of pituitary adenylate cyclase activating peptide (PACAP) and vasoactive intestinal peptide (VIP), and to inhibit the excessive activation of endothelial nitric oxide synthase or of inducible nitric oxide to a human patient in need thereof.

44. The method according to claim 43, wherein the somatostatin receptor agonist is selected from the group consisting of agonists of somatostatin receptor types 2 and 5.

45. The method according to claim 43, wherein the somatostatin receptor agonist is selected from the group consisting of agonists of somatostatin type 2A receptor.

46. The method according to claim 43, wherein the somatostatin receptor agonist is selected from the group consisting of non-peptide somatostatin agonists.

47. The method according to claim 43, wherein the somatostatin receptor agonist is octreotide acetate.

48. The method according to claim 43, wherein the somatostatin receptor agonist is lanreotide.

49. The method of preventing or treating gastroesophageal reflux disease (GERD), asthma-associated gastroesophageal reflux (GER), GER-associated asthma and asthma, or related disorders, which method comprises administering a composition comprising an effective amount of a somatostatin receptor agonist sufficient to produce parallel constrictive effects on the LES and the sphincter of Oddi to a human patient in need thereof.

50. The method according to claim 49, wherein the somatostatin receptor agonist is selected from the group consisting of agonists of somatostatin receptor types 2 and 5.

51. The method according to claim 49, wherein the somatostatin receptor agonist is selected from the group consisting of agonists of somatostatin type 2A receptor.

52. The method according to claim 49, wherein the somatostatin receptor agonist is selected from the group consisting of non-peptide somatostatin agonists.

53. The method according to claim 49, wherein the somatostatin receptor agonist is octreotide acetate.

54. The method according to claim 49, wherein the somatostatin receptor agonist is lanreotide.

55. The method of preventing or treating gastroesophageal reflux disease (GERD), asthma-associated gastroesophageal reflux (GER), GER-associated asthma and asthma, or related disorders, which method comprises administering a composition comprising an effective amount of a somatostatin receptor agonist sufficient to inhibit the synthesis and release of TNF-alpha, IL-1 beta, and INF-gamma by monocytes and T-cell lymphocytes to a human patient in need thereof.

56. The method according to claim 55, wherein the somatostatin receptor agonist is selected from the group consisting of agonists of somatostatin receptor types 2 and 5.

57. The method according to claim 55, wherein the somatostatin receptor agonist is selected from the group consisting of agonists of somatostatin type 2A receptor.

58. The method according to claim 55, wherein the somatostatin receptor agonist is selected from the group consisting of non-peptide somatostatin agonists.

59. The method according to claim 55, wherein the somatostatin receptor agonist is octreotide acetate.

60. The method according to claim 55, wherein the somatostatin receptor agonist is lanreotide.

61. The method of preventing or treating gastroesophageal reflux disease (GERD), asthma-associated gastroesophageal reflux (GER), GER-associated asthma and asthma, or related disorders, which method comprises administering a composition comprising an effective amount of a somatostatin receptor agonist sufficient to inhibit the activation of NF-kappaB and c-fos/AP-1 nuclear transcription factors to a human patient in need thereof.

62. The method according to claim 61, wherein the somatostatin receptor agonist is selected from the group consisting of agonists of somatostatin receptor types 2 and 5.

63. The method according to claim 61, wherein the somatostatin receptor agonist is selected from the group consisting of agonists of somatostatin type 2A receptor.

64. The method according to claim 61, wherein the somatostatin receptor agonist is selected from the group consisting of non-peptide somatostatin agonists.

65. The method according to claim 61, wherein the somatostatin receptor agonist is octreotide acetate.

66. The method according to claim 61, wherein the somatostatin receptor agonist is lanreotide.

67. The method of preventing or treating sleep apnea/hypopnea-associated episodes of hypoxemia and tissue hypoxia, which method comprises administering a composition comprising an effective amount of a somatostatin receptor agonist sufficient to inhibit the synthesis and/or secretion within the brain and/or the peripheral nervous system of excessive quantities of somnogenic peptides, and to inhibit or blunt the central and peripheral actions thereof, to a human patient in need thereof.

68. The method according to claim 67, wherein the somnogenic peptides are selected from the group consisting of growth hormone releasing hormone (GHRH), pituitary adenylate cyclase activating peptide (PACAP), vasoactive intestinal peptide (VIP), TNF-alpha, cortistatin, IL-6, and IL-1 beta.

69. The method according to claim 67, wherein the somatostatin receptor agonist is selected from the group consisting of agonists of somatostatin receptor types 2 and 5.

70. The method according to claim 67, wherein the somatostatin receptor agonist is selected from the group consisting of agonists of somatostatin type 2A receptor.

71. The method according to claim 67, wherein the somatostatin receptor agonist is selected from the group consisting of non-peptide somatostatin agonists.

72. The method according to claim 67, wherein the somatostatin receptor agonist is octreotide acetate.

73. The method according to claim 67, wherein the somatostatin receptor agonist is lanreotide.

74. The method of preventing or treating sleep apnea/hypopnea-associated gastroesophageal reflux disease (GERD), asthma-associated gastroesophageal reflux (GER), GER-associated asthma and asthma, or related disorders, which method comprises administering a composition comprising an effective amount of a somatostatin receptor agonist sufficient to inhibit the synthesis and/or secretion within the brain and/or the peripheral nervous system of excessive quantities of somnogenic peptides, and to inhibit or blunt the peripheral actions thereof, to a patient in need thereof.

75. The method according to claim 74, wherein the somnogenic peptides from the group consisting of pituitary adenylate cyclase activating peptide (PACAP), vasoactive intestinal peptide (VIP), TNF-alpha, and IL-1 beta.

76. The method according to claim 74, wherein the somatostatin receptor agonist is selected from the group consisting of agonists of somatostatin receptor types 2 and 5.

77. The method according to claim 74, wherein the somatostatin receptor agonist is selected from the group consisting of agonists of somatostatin type 2A receptor.

78. The method according to claim 74, wherein the somatostatin receptor agonist is selected from the group consisting of non-peptide somatostatin agonists.

79. The method according to claim 74, wherein the somatostatin receptor agonist is octreotide acetate.

80. The method according to claim 74, wherein the somatostatin receptor agonist is lanreotide.

81. The method of preventing or treating sleep apnea/hypopnea-associated and or sleep apnea/hypopnea-aggravated cardiomyopathy, cardioarrhythmia, and congestive heart failure, which method comprises administering a composition comprising an effective amount of a somatostatin receptor agonist sufficient to inhibit the secretion within the brain and/or the peripheral nervous system of excessive quantities of somnogenic peptides, and to inhibit or blunt the central and peripheral actions thereof, to a human patient in need thereof.

82. The methods according to claim 81, wherein the somnogenic peptides are selected from the group consisting of growth hormone-releasing hormone (GHRH), pituitary adenylate cyclase activating peptide (PACAP), vasoactive intestinal peptide (VIP) TNF-alpha, and IL-1 beta.

83. The method according to claim 81, wherein the somatostatin receptor agonist is selected from the group consisting of agonists of somatostatin receptor types 2 and 5.

84. The method according to claim 81, wherein the somatostatin receptor agonist is selected from the group consisting of agonists of somatostatin type 2A receptor.

85. The method according to claim 81, wherein the somatostatin receptor agonist is selected from the group consisting of non-peptide somatostatin agonists.

86. The method according to claim 81, wherein the somatostatin receptor agonist is octreotide acetate.

87. The method according to claim 81, wherein the somatostatin receptor agonist is lanreotide.

88. The method of preventing or treating sleep apnea/hypopnea-associated and/or sleep apnea/hypopnea-aggravated apparent life-threatening events (ALTE), referred to as a “near miss” for sudden infant death syndrome (SIDS), which method comprises administering a composition comprising an effective amount of a somatostatin receptor agonist sufficient to inhibit the secretion within the brain and/or the peripheral nervous system of excessive quantities of somnogenic peptides, and to inhibit or blunt the central and peripheral actions thereof, to a human patient in need thereof.

89. The method according to claim 88, wherein the somnogenic peptides are selected from the group consisting of growth hormone-releasing hormone (GHRH), pituitary adenylate cyclase activating peptide (PACAP) vasoactive intestinal peptide (VIP), TNF-alpha, and IL-1 beta.

90. The method according to claim 88, wherein the somatostatin receptor agonist is selected from the group consisting of agonists of somatostatin receptor types 2 and 5.

91. The method according to claim 88, wherein the somatostatin receptor agonist is selected from the group consisting of agonists of somatostatin type 2A receptor.

92. The method according to claim 88, wherein the somatostatin receptor agonist is selected from the group consisting of non-peptide somatostatin agonists.

93. The method according to claim 88, wherein the somatostatin receptor agonist is octreotide acetate.

94. The method according to claim 88, wherein the somatostatin receptor agonist is lanreotide.

95. The method of preventing or treating sleep apnea/hypopnea-associated and/or sleep apnea/hypopnea-aggravated neurologic disorders selected from the group consisting of median nerve compression neuropathy (carpal tunnel syndrome), motor and cognitive dysfunction post-cerebrovascular occlusion or hemorrhage, post-ischemia-reperfusion injury, cerebral arteriosclerosis, Alzheimer's disease, amyotrophyic lateral sclerosis (ALS), myasthenia gravis, central nervous system trauma, and alcoholism/post-alcoholism syndrome, which method comprises administering a composition comprising an effective amount of a somatostatin receptor agonist sufficient to inhibit the secretion within the brain and/or the peripheral nervous system of excessive quantities of somnogenic peptides, and to inhibit or blunt the central and peripheral actions thereof, to a human patient in need thereof.

96. The method according to claim 95, wherein the somnogenic peptides are selected from the group consisting of growth hormone-releasing hormone (GHRH), pituitary adenylate cyclase activating peptide (PACAP) vasoactive intestinal peptide (VIP), TNF-alpha, and IL-1 beta.

97. The method according to claim 96, wherein the somatostatin receptor agonist is selected from the group consisting of agonists of somatostatin receptor types 2 and 5.

98. The method according to claim 96, wherein the somatostatin receptor agonist is selected from the group consisting of agonists of somatostatin type 2A receptor.

99. The method according to claim 96, wherein the somatostatin receptor agonist is selected from the group consisting of non-peptide somatostatin agonists.

100. The method according to claim 96, wherein the somatostatin receptor agonist is octreotide acetate.

101. The method according to claim 96, wherein the somatostatin receptor agonist is lanreotide.

102. The method of preventing or treating disorders of excessive or undesired calpain activation arising from or aggravated by tissue hypoxia, selected from the group consisting of sleep apnea/hypopnea-induced tissue hypoxia, ischemia/reperfusion injury, direct physical injury, which method comprises administering a composition comprising an effective amount of a somatostatin receptor agonist sufficient to inhibit the activation of calpain in tissues with appropriate somatostatin receptors, to a human patient in need thereof.

103. The method according to claim 102, wherein the tissues are selected from the group consisting of tissues of the peripheral and central nervous systems, heart liver, kidney and gastrointestinal tract.

104. The method according to claim 102, wherein the somatostatin receptor agonist is selected from the group consisting of agonists of somatostatin receptor types 2 and 5.

105. The method according to claim 102, wherein the somatostatin receptor agonist is selected from the group consisting of agonists of somatostatin type 2A receptor.

106. The method according to claim 102, wherein the somatostatin receptor agonist is selected from the group consisting of non-peptide somatostatin agonists.

107. The method according to claim 102, wherein the somatostatin receptor agonist is octreotide acetate.

108. The method according to claim 102, wherein the somatostatin receptor agonist is lanreotide.
